SEQUENCE LISTING (11 sequences)

SEQ ID NO: 1 - protein, 120 aa, synthetic construct
  source 1..120: note = heavy chain variable region (VH) of the exemplary anti-APRIL antibody
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSS

SEQ ID NO: 2 - protein, 111 aa, synthetic construct
  source 1..111: note = light chain variable region (VL) of the exemplary anti-APRIL antibody
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIK

SEQ ID NO: 3 - protein, 7 aa, synthetic construct
  source 1..7: note = HCDR1
GYTFTDY

SEQ ID NO: 4 - protein, 6 aa, synthetic construct
  source 1..6: note = HCDR2
YPLRGS

SEQ ID NO: 5 - protein, 11 aa, synthetic construct
  source 1..11: note = HCDR3
HGAYYSNAFDY

SEQ ID NO: 6 - protein, 15 aa, synthetic construct
  source 1..15: note = LCDR1
RASESVDNDGIRFLH

SEQ ID NO: 7 - protein, 7 aa, synthetic construct
  source 1..7: note = LCDR2
RASTRAT

SEQ ID NO: 8 - protein, 9 aa, synthetic construct
  source 1..9: note = LCDR3
QQSNKDPYT

SEQ ID NO: 9 - protein, 446 aa, synthetic construct
  source 1..446: note = Heavy Chain
QVQLVQSGAEVKKPGASVKVSCKASGYTFTDYTIHWVRQATGQGLEWMGWIYPLRGSINYAQKFQGRVTMTADKSISTVYMELSSLRSEDTAVYFCARHGAYYSNAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 218 aa, synthetic construct
  source 1..218: note = Light Chain
EIVMTQSPATLSVSPGERATLSCRASESVDNDGIRFLHWYQQKPGQAPRLLIYRASTRATGIPARFSGSGSRTEFTLTISSLQSEDFAVYYCQQSNKDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 250 aa, Homo sapiens [sequence omitted]

(19) United States
(12) Patent Application Publication
(10) Pub. No.: US 2026/0152564 A1
(43) Pub. Date: Jun. 4, 2026

(54) METHODS OF TREATING SJOGREN'S DISEASE

(71) Applicant: OTSUKA AMERICA PHARMACEUTICAL, INC., Rockville, MD (US)

(72) Inventors: Jeffrey HAFKIN, Rockville, MD (US); Lokesh SHAH, Rockville, MD (US); Wei LI, Rockville, MD (US); Dorothee OBERDHAN, Rockville, MD (US); Michelle RICHARDSON, Rockville, MD (US)

(73) Assignee: OTSUKA AMERICA PHARMACEUTICAL, INC., Rockville, MD (US)

(21) Appl. No.: 19/406,608

(22) Filed: Dec. 2, 2025

Related U.S. Application Data
(60) Provisional application No. 63/727,587, filed on Dec. 3, 2024.
(60) Provisional application No. 63/906,848, filed on Oct. 28, 2025.

(51) Int. Cl. C07K 16/28; A61K 9/00; A61K 31/42; A61K 31/4706; A61K 31/519; A61K 31/52; A61K 31/573; A61K 38/00; A61P 37/06

(52) U.S. Cl. CPC C07K 16/2875; A61K 9/0019; A61K 31/42; A61K 31/4706; A61K 31/519; A61K 31/52; A61K 31/573; A61K 38/00; A61P 37/06; C07K 2317/565

(57) ABSTRACT

Methods of treating an autoimmune disease, e.g., Sjögren's disease, using APRIL inhibitor molecules are disclosed.

### CROSS REFERENCE TO RELATED APPLICATIONS

This application claims the benefit of the filing dates of U.S. Provisional Patent Application No. 63/727,587, filed Dec. 3, 2024 and U.S. Provisional Patent Application No. 63/906,848, filed Oct. 28, 2025, the contents of which are incorporated herein by reference in their entirety.

### SEQUENCE LISTING

The content of the electronically submitted sequence listing (Name: 5414_0120003_Sequencelisting_ST26.xml; Size: 13,729 bytes; and Date of Creation: Dec. 2, 2025) is incorporated herein by reference in its entirety.

### BACKGROUND

Sjögren's disease (Sjögren's) is estimated to be the second most common systemic autoimmune disease worldwide. Sjögren's disease is characterized by lymphocytic infiltration and tissue destruction of exocrine glands, primarily the salivary and lacrimal glands, which lead to xerostomia (dry mouth) and keratoconjunctivitis sicca (eye dryness), respectively. Approximately 40% of patients have extraglandular involvement, including involvement of the musculoskeletal, cutaneous, and respiratory systems and a substantial proportion of patients experience long-term disease progression leading to a wide variety of complications, including but not limited to lymphoma, pulmonary fibrosis, and renal failure, resulting in increased morbidity. Lymphoma is the primary factor affecting mortality in approximately 5% of patients, primarily B-cell lymphomas arising in the salivary glands that are the target of a chronic inflammatory autoimmune process. There are currently no treatments to modify the primary disease or progression of Sjögren's disease, resulting in a high, unmet need for the development of effective therapies for Sjögren's disease.

### SUMMARY

The present disclosure provides a method of treating Sjögren's disease in a subject in need thereof comprising administering to the subject an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

In some aspects, the present disclosure provides a method of treating Sjögren's disease in a population of subjects in need thereof comprising administering to the subjects an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain (VL) CDR1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

In some aspects, the VH of the anti-APRIL antibody or antigen-binding portion thereof comprises the amino acid sequence as set forth in SEQ ID NO: 1. In some aspects, the VL of the anti-APRIL antibody or antigen-binding portion thereof comprises the amino acid sequence as set forth in SEQ ID NO: 2. In some aspects, the antibody molecule further comprises a constant region linked to the VH and or VL.

In some aspects, the disclosure provides a method of treating Sjögren's disease in a subject in need thereof comprising administering to the subject an anti-APRIL antibody, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the disclosure provides a method of treating Sjögren's disease in a population of subjects in need thereof comprising administering to the subjects an anti-APRIL antibody, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the disclosure provides a method of treating Sjögren's disease in a subject in need thereof comprising administering to the subject an APRIL inhibitor, wherein the APRIL inhibitor comprises Zigakibart/BION-1301, Atacicept, or Povetacicept. In some aspects, the disclosure provides a method of treating Sjögren's disease in a population of subjects in need thereof comprising administering to the subjects an APRIL inhibitor, wherein the APRIL inhibitor comprises Zigakibart/BION-1301, Atacicept, or Povetacicept. In some aspects, the APRIL inhibitor comprises Zigakibart/BION-1301. In some aspects, the APRIL inhibitor comprises a fusion protein. In some aspects, the APRIL inhibitor comprises Povetacicept. In some aspects, the APRIL inhibitor comprises Atacicept.

In some aspects, the subject or each of the subjects for the present methods has a range of ages between 18 years old and 75 years old. In some aspects, the subject or each of the subjects for the present methods has a European League against Rheumatism (EULAR) Sjögren's syndrome disease activity index (ESSDAI) score equal to or higher than 1, 2, 3, 4, 5, 6, 7, 8, 9, 10, 11, 12, 13, 14, 15, 16, 17, 18, 19, or 20. In some aspects, the subject or each of the subjects has an ESSDAI score equal to or higher than 5. In some aspects, the subject or each of the subjects has an ESSDAI score between 1 and 5. In some aspects, the subject or each of the subjects has an ESSDAI score between 1 and 10. In some aspects, the subject or each of the subjects has an ESSDAI score equal to or higher than 10. In some aspects, the subject or each of the subjects has an ESSDAI score between 10 and 30, between 10 and 20, between 10 and 25, between 10 and 15, or between 10 and 40.

In some aspects, the subject or each of the subjects for the present methods is seropositive for an anti-Sjögren's-syndrome-related antigen A (anti-SSA/Ro) antibody, an anti-Sjögren's-syndrome-related antigen B (anti-SSB) antibody, or rheumatoid factor. In some aspects, the anti-SSA/Ro antibody comprises an anti-Ro52 antibody or anti-Ro60 antibody. In some aspects, the subject or each of the subjects has a stimulated whole salivary flow rate of equal to or higher than 0.05 mL/min. In some aspects, the subject or each of the subjects has a serum IgG concentration greater than 900 mg/dL.

In some aspects, the anti-APRIL antibody or the antigen-binding portion thereof or the APRIL inhibitor is administered subcutaneously. In some aspects, the anti-APRIL antibody or the antigen-binding portion thereof or the APRIL inhibitor is administered at a fixed dose between 10 mg and 1000 mg. In some aspects, the anti-APRIL antibody or the antigen-binding portion thereof or the APRIL inhibitor is administered at a fixed dose of 400 mg once every 4 weeks. In some aspects, the anti-APRIL antibody or the antigen binding portion thereof or the APRIL inhibitor is administered twice, three times, four times, five times, six times, seven times, eight times, nine times, 10 times, 11 times, 12 times, 13 times, 14 times, or 15 times. In some aspects, the anti-APRIL antibody or the antigen binding portion thereof or the APRIL inhibitor is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.

In some aspects, the present methods further comprise administering to the subject or each of the subjects a standard of care (SOC) therapy. In some aspects, the subject or each of the subjects received a SOC therapy prior to the administration. In some aspects, the subject or each of the subjects received a SOC therapy for at least 30 days prior to the administration. In some aspects, the SOC therapy comprises (i) hydroxychloroquine, (ii) a corticosteroid (e.g., prednisone or prednisolone), and/or (iii) any one of methotrexate, leflunomide, or azathioprine. In some aspects, the SOC therapy comprises hydroxychloroquine, a corticosteroid (e.g., prednisone or prednisolone), methotrexate, leflunomide, azathioprine, or any combination thereof. In some aspects, the hydroxychloroquine is or has been administered at a dose of ≤400 mg/day. In some aspects, the hydroxychloroquine is or has been administered at a dose of between about 10 mg/day and about 400 mg/day, about 100 mg/day and about 400 mg/day, about 200 mg/day and about 400 mg/day, about 300 mg/day and about 400 mg/day, about 10 mg/day and about 300 mg/day, about 100 mg/day and about 300 mg/day, about 200 mg/day and about 300 mg/day, about 10 mg/day and about 200 mg/day, about 100 mg/day and about 200 mg/day, or about 10 mg/day and about 100 mg/day. In some aspects, the hydroxychloroquine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 150 mg/day, about 200 mg/day, about 250 mg/day, about 300 mg/day, about 350 mg/day, or about 400 mg/day.

In some aspects, the corticosteroid (e.g., prednisone or prednisolone) is or has been administered at a dose of ≤10 mg/day. In some aspects, the corticosteroid (e.g., prednisone or prednisolone) is or has been administered at a dose of between about 1 mg/day and about 10 mg/day, about 2 mg/day and about 10 mg/day, about 4 mg/day and about 10 mg/day, about 6 mg/day and about 10 mg/day, about 8 mg/day and about 10 mg/day, about 2 mg/day and about 8 mg/day, about 4 mg/day and about 8 mg/day, about 6 mg/day and about 8 mg/day, about 2 mg/day and about 6 mg/day, about 4 mg/day and about 6 mg/day.

In some aspects, the corticosteroid (e.g., prednisone or prednisolone) is or has been administered at a dose of about 1 mg/day, about 2 mg/day, about 3 mg/day, about 4 mg/day, about 5 mg/day, about 6 mg/day, about 7 mg/day, about 8 mg/day, about 9 mg/day, or about 10 mg/day.

In some aspects, the methotrexate is or has been administered at a dose of ≤25 mg/week. In some aspects, the methotrexate is or has been administered at a dose of about 1 mg/week, about 2 mg/week, about 3 mg/week, about 4 mg/week, about 5 mg/week, about 6 mg/week, about 7 mg/week, about 8 mg/week, about 9 mg/week, about 10 mg/week, about 11 mg/week, about 12 mg/week, about 13 mg/week, about 14 mg/week, about 15 mg/week, about 16 mg/week, about 17 mg/week, about 18 mg/week, about 19 mg/week, about 20 mg/week, about 21 mg/week, about 22 mg/week, about 23 mg/week, about 24 mg/week, or about 25 mg/week.

In some aspects, the leflunomide is or has been administered at a dose of ≤20 mg daily. In some aspects, the leflunomide is or has been administered at a dose of about 1 mg daily, about 2 mg daily, about 3 mg daily, about 4 mg daily, about 5 mg daily, about 6 mg daily, about 7 mg daily, about 8 mg daily, about 9 mg daily, about 10 mg daily, about 11 mg daily, about 12 mg daily, about 13 mg daily, about 14 mg daily, about 15 mg daily, about 16 mg daily, about 17 mg daily, about 18 mg daily, about 19 mg daily, or about 20 mg daily.

In some aspects, the azathioprine is or has been administered at a dose of ≤150 mg/day. In some aspects, the azathioprine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, or about 150 mg/day.

In some aspects, the corticosteroid for the present disclosure comprises prednisone, prednisolone, cortisone, hydrocortisone, methylprednisolone, hydrocortisone acetate, cortisone acetate, tixocortol pivalate, acetonides, amcinonide, budesonide, desonide, fluocinolone acetonide, fluocinonide, halcinonide, triamcinolone acetonide, Deflazacort (0-isopropylidene derivative), beclometasone, betamethasone, dexamethasone, fluocortolone, halometasone, mometasone, esters, halogenated, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

In some aspects, after the administration, the subject or each of the subjects for the present method exhibits a decrease in EULAR Sjögren's syndrome disease activity index (ESSDAI) that is equal to or higher than 1 point, 2 points, or 3 points from baseline at 28 weeks after the initial administration. In some aspects, after the administration, the subject or each of the subjects exhibits a decrease in EULAR Sjögren's syndrome disease activity index (ESSDAI) that is equal to or higher than 3 points from baseline at 28 weeks after the initial administration. In some aspects, after the administration, the subject or each of the subjects exhibits a decrease in EULAR Sjögren's Syndrome Patient Reported Index ESSPRI that is equal to or higher than 1 point from the baseline at 28 weeks after the initial administration. In some aspects, after the administration, the subject or each of the subjects exhibits one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

In some aspects, the subject or each of the subjects for the present disclosure exhibits a decrease in serum anti-drug antibody (ADA). In some aspects, the treatment is terminated if the subject exhibits an increase in serum ADA, or no decrease in serum ADA. In some aspects, the subject or each of the subjects exhibits an incidence of treatment emergent adverse events (TEAEs) graded by severity, clinical laboratory tests, vital sign measurements, physical examinations, injection site reactions, or any combination thereof. In some aspects, the subject or each of the subjects exhibits:
a) reduction of serum IgG;
b) reduction in circulating peripheral immune cells;
c) reversible reductions in total serum IgA;
d) reversible reductions in total serum IgM and IgG;
e) reduction in mononuclear cells staining positive for IgA, IgG, and/or IgM in GALT and/or tonsils; or
f) any combination thereof.

In some aspects, the subject or each of the subjects exhibits reduction of serum IgG at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reduction of serum IgG at a level between about 60% to about 95%. In some aspects, the subject or each of the subjects exhibits reduction in circulating peripheral immune cells at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reversible reductions in total serum IgA at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reversible reductions in total serum IgM and IgG at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reduction in mononuclear cells staining positive for IgA, IgG, and/or IgM in GALT and/or tonsils at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits a change in (i) free APRIL concentrations, (ii) immunoglobulins including autoantibodies; (iii) characteristic of Sjögren's B-cell receptor; (iv) repertoire, inflammatory factors, and gene; (v) expression signatures over time; or (vi) any combination thereof.

In some aspects, after the administration, the subject or each of the subjects exhibits a decrease in EULAR Sjögren's syndrome disease activity index (ESSDAI) that is equal to or higher than 1 point, 2 points, or 3 points from the baseline at 52 weeks after the initial administration. In some aspects, after the administration, the subject or each of the subjects exhibits a decrease in EULAR Sjögren's syndrome disease activity index (ESSDAI) that is equal to or higher than 3 points from the baseline at 52 weeks after the initial administration. In some aspects, after the administration, the subject or each of the subjects exhibits a decrease in EULAR Sjögren's syndrome disease activity index (ESSDAI) that is equal to or higher than 3 points from the baseline at 52 weeks after the initial administration. In some aspects, after the administration, the subject or each of the subjects exhibits a decrease in EULAR Sjögren's Syndrome Patient Reported Index ESSPRI that is equal to or higher than 1 point from the baseline at 52 weeks after the initial administration.

In some aspects, after the administration, the subject or each of the subjects exhibits one or more of the following:
h. a decrease in individual ESSDAI domains from the baseline at 52 weeks after the initial administration;
i. an increase in the salivary flow rate and/or tear flow rate, e.g., 0.1 mL/min or higher from the baseline at 52 weeks after the initial administration;
j. a decrease in Physician's global assessment (PhGA) score of disease activity from the baseline at 52 weeks after the initial administration;
k. a decrease in Patient's global assessment (PaGA) score of participant outcomes from the baseline at 52 weeks after the initial administration;
l. a decrease in Functional Assessment of Chronic Illness Therapy-Fatigue (FACIT-F) score from the baseline at 52 weeks after the initial administration;
m. an improvement in 36-Item Short Form Health Survey questionnaire version 2 (SF-36v2) physical and mental composite score from the baseline at 52 weeks after the initial administration; and
n. an improvement in Patient-reported Sjögren's disease diary from the baseline at 52 weeks after the initial administration.

In some aspects, the subject or each of the subjects does not have another active autoimmune rheumatic disease. In some aspects, the subject or each of the subjects has not received a B-cell depleting therapy. In some aspects, the subject or each of the subjects has not received a B-cell depleting therapy within 12 months prior to the administration or if received, the B-cell count is less than the lower limit of normal or baseline value prior to the receipt of previous B cell-depleting therapy (whichever is lower). In some aspects, the subject or each of the subjects has not previously treated with one or more of the following within at least 6 months prior to the administration:
a. belimumab, abatacept, anti-tumor necrosis factor alpha biologic agents,
b. immunoglobulins, plasmapheresis; intravenous (IV) or oral cyclophosphamide, and
c. mycophenolate mofetil, IV or oral cyclosporine A or any other immunosuppressants.

In some aspects, the subject or each of the subjects does not exhibit the following laboratory values at screening:
a. Hemoglobin levels <8.0 g/dL;
b. White blood cells (WBC) count <2.0×103/μL;
c. Platelet count <100×103/μL;
d. Absolute neutrophil count (ANC)<0.8×103/μL; and/or
e. Estimated glomerular filtration rate (eGFR) calculated using the 2021 Chronic Kidney Disease-Epidemiology Collaboration (CKD-EPI) serum creatinine eGFR formula <45 mL/min/1.73 m2.

In some aspects, the subject or each of the subjects does not exhibit active viral, bacterial, or other infections requiring systemic treatment at the time of screening or randomization, or history of recurrent clinically significant infection. In some aspects, the subject or each of the subjects does not have history of a previous hypersensitivity or severe allergic reaction with generalized urticaria, angioedema, or anaphylaxis to any of the ingredients of the anti-APRIL antibody or the APRIL inhibitor SC injection formulation. In some aspects, the subject or each of the subjects has not had a history of major organ, hematopoietic stem cell, or bone marrow transplant. In some aspects, the subject or each of the subjects does not take a medication known to cause dry mouth/eyes as a regular and major side effect, and which have not been on a stable dose for at least 30 days prior to the administration, or any anticipated change in the treatment regimen during the course of the administration. In some aspects, the subject or each of the subjects does not use a topical ocular prescription medication (excluding artificial tears, gels, lubricants) that have not been on a stable dose for at least 90 days prior to administration, or any anticipated change in the treatment regimen during the course of the administration. In some aspects, the subject or each of the subjects does not have a history of primary or secondary immunodeficiency, including a positive human immunodeficiency virus (HIV) test result.

In some aspects, the subject or each of the subjects has not a history of malignancy of any organ system (other than localized basal cell carcinoma of the skin or in situ cervical cancer), treated or untreated, within the past 5 years, regardless of whether there is evidence of local recurrence or metastases. In some aspects, the subject or each of the subjects does not have sarcoidosis. In some aspects, the subject or each of the subjects has not have a history of fibromyalgia. In some aspects, the subject or each of the subjects does not have uncontrolled type 2 diabetes, as evidenced by a screening hemoglobin A1c (HbA1c) value >8%. In some aspects, psychiatric or additional physical condition that interferes with the administration. In some aspects, the subject or each of the subjects does not have a positive serology for hepatitis B surface antigen (HBsAg). In some aspects, the subject or each of the subjects does not have hepatitis C. In some aspects, the subject or each of the subjects does not have positive hepatitis C antibody and hepatitis C virus (HCV)-ribonucleic acid (RNA) at the administration or prior to administration except if the subject or each of the subjects has chronic hepatitis C, but has completed HCV anti-viral treatment and is HCV-RNA negative at least 12 weeks after the anti-viral treatment before the administration. In some aspects, the subject or each of the subjects does not have active tuberculosis infection. In some aspects, the subject or each of the subjects is not pregnant or nursing (lactating).

In some aspects, the subject or each of the subjects after the administration exhibits mean AUCtau of about 4000 day*μg/ml to about 6000 day*μg/ml, e.g., 4000 day*μg/ml, 4100 day*μg/ml, 4200 day*μg/ml, 4300 day*μg/ml, 4400 day*μg/ml, 4500 day*μg/ml, 4600 day*μg/ml, 4700 day*μg/ml, 4800 day*μg/ml, 4900 day*μg/ml, 5000 day*μg/ml, 5100 day*μg/ml, 5200 day*μg/ml, 5300 day*μg/ml, 5400 day*μg/ml, 5500 day*μg/ml, 5600 day*μg/ml, 5700 day*μg/ml, 5800 day*μg/ml, 5900 day*μg/ml, or 6000 day*μg/ml. In some aspects, the subject or each of the subjects after the administration exhibits mean (SD) AUCtau of about 4830 (2780) day*μg/mL. In some aspects, the subject or each of the subjects after the administration exhibits mean (SD) Cmax of about 100 μg/ml to about 300 μg/ml, e.g., 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, 290 μg/ml, 300 μg/ml. In some aspects, the subject or each of the subjects after the administration exhibits mean (SD) Cmax of about 194(100) μg/ml. In some aspects, the subject or each of the subjects after the administration exhibits mean Ctrough of about 100 μg/ml to about 200 μg/ml, e.g., 110 μg/ml, 120 μg/ml, 130 μg/ml, 140 μg/ml, 150 μg/ml, 160 μg/ml, 170 μg/ml, 180 μg/ml, 190 μg/ml or 200 μg/ml. In some aspects, the subject or each of the subjects after the administration exhibits mean (SD) Ctrough of about 141 (94) g/mL. In some aspects, the subject or each of the subjects after the administration exhibits mean (SD) Ctrough of about 141 (94) g/mL, mean (SD) Cmax of about 194(100) g/ml, mean (SD) AUCtau of about 4830 (2780) day*μg/mL, or any combination thereof. In some aspects, the subject or each of the subjects does not have a mixed connective-tissue disease or Sjögren overlap syndrome.

In some aspects, the present disclosure provides a pharmaceutical composition comprising: (a) an anti-APRIL antibody and (b) one or more ingredients comprising L-arginine, L-glutamic acid, L-histidine, L-histidine monohydrochloride, polysorbate 80, and/or sorbitol. In some aspects the present disclosure provides a pharmaceutical composition comprising: (a) an anti-APRIL antibody and (b) one or more ingredients comprising L-arginine, L-glutamic acid, L-histidine, polysorbate 80, and/or sorbitol.

In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises zigakibart/BION-1301.

In some aspects, the pharmaceutical composition is a liquid formulation. In some aspects, the pharmaceutical composition is a liquid formulation for subcutaneous injection. In some aspects, the pharmaceutical composition is contained within a syringe. In some aspects, the pharmaceutical composition is contained within an autoinjector. In some aspects, the pH of the pharmaceutical composition is between about 5 and about 7, e.g., about 5.0, about 5.25, about 5.5, about 5.75, about 6.0, about 6.25, about 6.5, about 6.75, or about 7.0. In some aspects, the pH is about 6.2. In some aspects, the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL. In some aspects, the total volume of the composition is about 2 mL. In some aspects, the pharmaceutical composition comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol. In some aspects, the pharmaceutical composition further comprises water for injection. In some aspects, the pH of the composition is about 6.2. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2. In some aspects, the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol. In some aspects, the pharmaceutical composition further comprises a trace amount of a chloride ion. In some aspects, the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg. In some aspects, the composition comprises about 400 mg of the anti-APRIL antibody. In some aspects, the pharmaceutical composition further comprises an overfill. In some aspects, the overfill comprises about 0.0125 mL to about 0.5 mL. In some aspects, the overfill comprises about 0.025 mL to about 0.5 mL, about 0.025 to about 0.05 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL. In some aspects, the overfill comprises about 0.0125 mL, about 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.

In some aspects, the present disclosure provides a pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the present disclosure provides a pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody in the composition is about 400 mg. In some aspects, the anti-APRIL antibody comprises Sibeprenlimab or an antigen binding portion thereof.

In some aspects, the present disclosure provides a method of treating a disease or disorder associated with APRIL, e.g., Sjögren's disease, in a subject in need thereof comprising administering to the subject the pharmaceutical composition.

In some aspects, the present disclosure provides a method of producing a pharmaceutical composition comprising admixing one or more ingredients in the pharmaceutical composition.

Other features, objects, and advantages of the compositions and methods herein will be apparent from the description and drawings, and from the claims.

### Exemplary Aspects

[A1]A method of treating Sjögren's disease in a subject in need thereof comprising administering to the subject an APRIL inhibitor thereof.
[A2]A method of treating Sjögren's disease in a population of subjects in need thereof comprising administering to the subject an APRIL inhibitor thereof.
[A3] The method of A1 or A2, wherein the APRIL inhibitor comprises an anti-APRIL antibody or an antigen-binding portion thereof.
[A4] The method of A3, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein: the VH CDR1 comprises the amino acid sequence set forth in SEQ ID NO: 3;
   the VH CDR2 comprises the amino acid sequence set forth in SEQ ID NO: 4;
   the VH CDR3 comprises the amino acid sequence set forth in SEQ ID NO: 5;
   the VL CDR1 comprises the amino acid sequence set forth in SEQ ID NO: 6;
   the VL CDR2 comprises the amino acid sequence set forth in SEQ ID NO: 7; and
   the VL CDR3 comprises the amino acid sequence set forth in SEQ ID NO: 8.
[A5] The method of A4, wherein the anti-APRIL antibody comprises a VH comprising the amino acid sequence set forth in SEQ ID NO: 1 and a VL comprising the amino acid sequence set forth in SEQ ID NO: 2.
[A6] The method of A4 or A5, wherein the anti-APRIL antibody comprises a heavy chain comprising the amino acid sequence set forth in SEQ ID NO: 9 and a light chain comprising the amino acid sequence set forth in SEQ ID NO: 10.
[A7] The method of any one of A3 to A6, wherein the anti-APRIL antibody comprises sibeprenlimab.
[A8] The method of A3, wherein the anti-APRIL antibody comprises zigakibart (BION-1301).
[A9] The method of A3, wherein the anti-APRIL antibody is any one of the antibodies disclosed in WO2010/100056, WO2015/034364, WO2016/110587, WO2021/243298 or WO2024/105549.
[A10] The method of A1 or A2, wherein the APRIL inhibitor comprises a TACI-fusion protein.
[A11] The method of A10, wherein the TACI-fusion protein comprises atacicept or povetacicept.
[A12] The method of A10, wherein the TACI-fusion protein is any one of the proteins disclosed in WO2002/094852, WO2021/226551, WO2022/236335, WO2024/123675, or WO2024/077018.
[A13] The method of A1 to A12, wherein the subject or each of the subjects has a range of ages between 18 years old and 75 years old.
[A14] The method of any one of A1 to A13, wherein the subject or each of the subjects has a European League against Rheumatism (EULAR) Sjögren's syndrome disease activity index (ESSDAI) score equal to or higher than 1, 2, 3, 4, 5, 6, 7, 8, 9, 10, 11, 12, 13, 14, 15, 16, 17, 18, 19, or 20.
[A15] The method of A1 to A14, wherein the subject or each of the subjects has an ESSDAI score equal to or higher than 5.
[A16] The method of A1 to A14, wherein the subject or each of the subjects has an ESSDAI score between 1 and 5.
[A17] The method of A1 to A14, wherein the subject or each of the subjects has an ESSDAI score between 1 and 10.
[A18] The method of A1 to A14, wherein the subject or each of the subjects has an ESSDAI score equal to or higher than 10.
[A19] The method of A1 to A14, wherein the subject or each of the subjects has an ESSDAI score between 10 and 30, between 10 and 20, between 10 and 25, between 10 and 15, or between 10 and 40.
[A20] The method of A1 to A19, wherein the subject or each of the subjects is seropositive for an anti-Sjögren's-syndrome-related antigen A (anti-SSA/Ro) antibody, an anti-Sjögren's-syndrome-related antigen B (anti-SSB) antibody, or rheumatoid factor.
[A21] The method of A20, wherein the anti-SSA/Ro antibody comprises an anti-Ro52 antibody or anti-Ro60 antibody.
[A22] The method of A1 to A21, wherein the subject or each of the subjects has a stimulated whole salivary flow rate of equal to or higher than 0.05 mL/min.
[A23] The method of A1 to A22, wherein the subject or each of the subjects has a serum IgG concentration greater than 900 mg/dL.
[A24] The method of A1 to A23, wherein the anti-APRIL antibody or the antigen-binding portion thereof is administered subcutaneously.
[A25] The method of A1 to A24, wherein the anti-APRIL antibody or the antigen-binding portion thereof is administered at a fixed dose between 10 mg and 1000 mg.
[A26] The method of A1 to A25, wherein the anti-APRIL antibody or the antigen-binding portion thereof is administered at a fixed dose of 400 mg once every 4 weeks.
[A27] The method of A1 to A26, wherein the anti-APRIL antibody or the antigen binding portion thereof is administered twice, three times, four times, five times, six times, seven times, eight times, nine times, 10 times, 11 times, 12 times, 13 times, 14 times, or 15 times.
[A28] The method of A26 or A27, wherein the anti-APRIL antibody or the antigen binding portion thereof is administered for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks.
[A29] The method of A1 to A28, further comprising administering to the subject or each of the subjects a standard of care (SOC) therapy.
[A30] The method of A1 to A29, wherein the subject or each of the subjects received a SOC therapy prior to the administration.
[A31] The method of A30, wherein the subject or each of the subjects received a SOC therapy for at least 30 days prior to the administration.
[A32] The method of A29 to A31, wherein the SOC therapy comprises (i) hydroxychloroquine, (ii) a corticosteroid (e.g., prednisone or prednisolone), and/or (iii) any one of methotrexate, leflunomide, or azathioprine.
[A33] The method of A29 to A31, wherein the SOC therapy comprises hydroxychloroquine, a corticosteroid (e.g., prednisone or prednisolone), methotrexate, leflunomide, azathioprine, or any combination thereof.
[A34] The method of A32 or A33, wherein the hydroxychloroquine is or has been administered at a dose of ≤400 mg/day.
[A35] The method of A34, wherein the hydroxychloroquine is or has been administered at a dose of between about 10 mg/day and about 400 mg/day, about 100 mg/day and about 400 mg/day, about 200 mg/day and about 400 mg/day, about 300 mg/day and about 400 mg/day, about 10 mg/day and about 300 mg/day, about 100 mg/day and about 300 mg/day, about 200 mg/day and about 300 mg/day, about 10 mg/day and about 200 mg/day, about 100 mg/day and about 200 mg/day, or about 10 mg/day and about 100 mg/day.
[A36] The method of A34, wherein the hydroxychloroquine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 150 mg/day, about 200 mg/day, about 250 mg/day, about 300 mg/day, about 350 mg/day, or about 400 mg/day.
[A37] The method of A32 to A36, wherein the corticosteroid (e.g., prednisone or prednisolone) is or has been administered at a dose of ≤10 mg/day.
[A38] The method of A37, wherein the corticosteroid (e.g., prednisone or prednisolone) is or has been administered at a dose of between about 1 mg/day and about 10 mg/day, about 2 mg/day and about 10 mg/day, about 4 mg/day and about 10 mg/day, about 6 mg/day and about 10 mg/day, about 8 mg/day and about 10 mg/day, about 2 mg/day and about 8 mg/day, about 4 mg/day and about 8 mg/day, about 6 mg/day and about 8 mg/day, about 2 mg/day and about 6 mg/day, about 4 mg/day and about 6 mg/day.
[A39] The method of A37 or A38, wherein the corticosteroid (e.g., prednisone or prednisolone) is or has been administered at a dose of about 1 mg/day, about 2 mg/day, about 3 mg/day, about 4 mg/day, about 5 mg/day, about 6 mg/day, about 7 mg/day, about 8 mg/day, about 9 mg/day, or about 10 mg/day.
[A40] The method of A32 to A39, wherein the methotrexate is or has been administered at a dose of ≤25 mg/week.
[A41] The method of A40, wherein the methotrexate is or has been administered at a dose of about 1 mg/week, about 2 mg/week, about 3 mg/week, about 4 mg/week, about 5 mg/week, about 6 mg/week, about 7 mg/week, about 8 mg/week, about 9 mg/week, about 10 mg/week, about 11 mg/week, about 12 mg/week, about 13 mg/week, about 14 mg/week, about 15 mg/week, about 16 mg/week, about 17 mg/week, about 18 mg/week, about 19 mg/week, about 20 mg/week, about 21 mg/week, about 22 mg/week, about 23 mg/week, about 24 mg/week, or about 25 mg/week.
[A42] The method of A32 to A41, wherein the leflunomide is or has been administered at a dose of ≤20 mg daily.
[A43] The method of A42, wherein the leflunomide is or has been administered at a dose of about 1 mg daily, about 2 mg daily, about 3 mg daily, about 4 mg daily, about 5 mg daily, about 6 mg daily, about 7 mg daily, about 8 mg daily, about 9 mg daily, about 10 mg daily, about 11 mg daily, about 12 mg daily, about 13 mg daily, about 14 mg daily, about 15 mg daily, about 16 mg daily, about 17 mg daily, about 18 mg daily, about 19 mg daily, about 20 mg daily.
[A44] The method of A32 to A43, wherein the azathioprine is or has been administered at a dose of ≤150 mg/day.
[A45] The method of A44, wherein the azathioprine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, or about 150 mg/day.
[A46] The method of A32 to A45, wherein the corticosteroid comprises prednisone, prednisolone, cortisone, hydrocortisone, methylprednisolone, hydrocortisone acetate, cortisone acetate, tixocortol pivalate, acetonides, amcinonide, budesonide, desonide, fluocinolone acetonide, fluocinonide, halcinonide, triamcinolone acetonide, Deflazacort (0-isopropylidene derivative), beclometasone, betamethasone, dexamethasone, fluocortolone, halometasone, mometasone, esters, halogenated, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.
[A47] The method of A32 to A45, wherein the corticosteroid comprises prednisone or prednisolone.
[A48] The method of A1 to A47, wherein after the administration, the subject or each of the subjects exhibits a decrease in EULAR Sjögren's syndrome disease activity index (ESSDAI) that is equal to or higher than 1 point, 2 points, or 3 points from baseline at 28 weeks after the initial administration.
[A49] The method of A1 to A47, wherein after the administration, the subject or each of the subjects exhibits a decrease in EULAR Sjögren's syndrome disease activity index (ESSDAI) that is equal to or higher than 3 points from baseline at 28 weeks after the initial administration.
[A50] The method of A1 to A47, wherein after the administration, the subject or each of the subjects exhibits a decrease in EULAR Sjögren's Syndrome Patient Reported Index ESSPRI that is equal to or higher than 1 point from the baseline at 28 weeks after the initial administration.
[A51] The method of A1 to A50, wherein after the administration, the subject or each of the subjects exhibits one or more of the following:
   i. a decrease in individual ESSDAI domains from the baseline at 28 weeks after the initial administration;
   ii. an increase in the salivary flow rate and/or tear flow rate, e.g., 0.1 mL/min or higher from the baseline at 28 weeks after the initial administration;
   iii. a decrease in Physician's global assessment (PhGA) score of disease activity from the baseline at 28 weeks after the initial administration;
   iv. a decrease in Patient's global assessment (PaGA) score of participant outcomes from the baseline at 28 weeks after the initial administration;
   v. a decrease in Functional Assessment of Chronic Illness Therapy-Fatigue (FACIT-F) score from the baseline at 28 weeks after the initial administration;
   vi. an improvement in 36-Item Short Form Health Survey questionnaire version 2 (SF-36v2) physical and mental composite score from the baseline at 28 weeks after the initial administration; and
   vii. an improvement in Patient-reported Sjögren's disease diary from the baseline at 28 weeks after the initial administration.
[A52] The method of A1 to A51, wherein the subject or each of the subjects exhibits a decrease in serum anti-drug antibody (ADA).
[A53] The method of A1 to A52, wherein the treatment is terminated if the subject exhibits an increase in serum ADA, or no decrease in serum ADA.
[A54] The method of A1 to A53, wherein the subject or each of the subjects exhibits an incidence of treatment emergent adverse events (TEAEs) graded by severity, clinical laboratory tests, vital sign measurements, physical examinations, injection site reactions, or any combination thereof.
[A55] The method of A1 to A54, wherein the subject or each of the subjects exhibits:
   i. reduction of serum IgG;
   ii. reduction in circulating peripheral immune cells;
   iii. reversible reductions in total serum IgA;
   iv. reversible reductions in total serum IgM and IgG;
   v. reduction in mononuclear cells staining positive for IgA, IgG, and/or IgM in GALT and/or tonsils; or
   vi. any combination thereof.
[A56] The method of A1 to A55, wherein the subject or each of the subjects exhibits reduction of serum IgG at a level of at least about 10%, about 20%, about 30%, about 40%, about 50%, about 60%, about 70%, about 80%, about 90%, about 95%, or about 99% compared to the serum IgG level prior to the administration.
[A57] The method of A1 to A56, wherein the subject or each of the subjects exhibits reduction of serum IgG at a level between about 60% to about 95%.
[A58] The method of A1 to A57, wherein the subject or each of the subjects exhibits reduction in circulating peripheral immune cells at a level of at least about 10%, about 20%, about 30%, about 40%, about 50%, about 60%, about 70%, about 80%, about 90%, about 95%, or about 99% compared to the serum IgG level prior to the administration.
[A59] The method of A1 to A58, wherein the subject or each of the subjects exhibits reversible reductions in total serum IgA at a level of at least about 10%, about 20%, about 30%, about 40%, about 50%, about 60%, about 70%, about 80%, about 90%, about 95%, or about 99% compared to the serum IgG level prior to the administration.
[A60] The method of A1 to A59, wherein the subject or each of the subjects exhibits reversible reductions in total serum IgM and IgG at a level of at least about 10%, about 20%, about 30%, about 40%, about 50%, about 60%, about 70%, about 80%, about 90%, about 95%, or about 99% compared to the serum IgG level prior to the administration.
[A61] The method of A1 to A60, wherein the subject or each of the subjects exhibits reduction in mononuclear cells staining positive for IgA, IgG, and/or IgM in GALT and/or tonsils at a level of at least about 10%, about 20%, about 30%, about 40%, about 50%, about 60%, about 70%, about 80%, about 90%, about 95%, or about 99% compared to the serum IgG level prior to the administration.
[A62] The method of A1 to A61, wherein the subject or each of the subjects exhibits a change in (i) free APRIL concentrations, (ii) immunoglobulins including autoantibodies; (iii) characteristic of Sjögren's B-cell receptor; (iv) repertoire, inflammatory factors, and gene; (v) expression signatures over time; or (vi) any combination thereof.
[A63] The method of A1 to A62, wherein after the administration, the subject or each of the subjects exhibits a decrease in EULAR Sjögren's syndrome disease activity index (ESSDAI) that is equal to or higher than 1 point, 2 points, or 3 points from the baseline at 52 weeks after the initial administration.
[A64] The method of A1 to A62, wherein after the administration, the subject or each of the subjects exhibits a decrease in EULAR Sjögren's syndrome disease activity index (ESSDAI) that is equal to or higher than 3 points from the baseline at 52 weeks after the initial administration.
[A65] The method of A1 to A64, wherein after the administration, the subject or each of the subjects exhibits a decrease in EULAR Sjögren's syndrome disease activity index (ESSDAI) that is equal to or higher than 3 points from the baseline at 52 weeks after the initial administration.
[A66] The method of A1 to A65, wherein after the administration, the subject or each of the subjects exhibits a decrease in EULAR Sjögren's Syndrome Patient Reported Index ESSPRI that is equal to or higher than 1 point from the baseline at 52 weeks after the initial administration.
[A67] The method of A1 to A66, wherein after the administration, the subject or each of the subjects exhibits one or more of the following:
   i. a decrease in individual ESSDAI domains from the baseline at 52 weeks after the initial administration;
   ii. an increase in the salivary flow rate and/or tear flow rate, e.g., 0.1 mL/min or higher from the baseline at 52 weeks after the initial administration;
   iii. a decrease in Physician's global assessment (PhGA) score of disease activity from the baseline at 52 weeks after the initial administration;
   iv. a decrease in Patient's global assessment (PaGA) score of participant outcomes from the baseline at 52 weeks after the initial administration;
   v. a decrease in Functional Assessment of Chronic Illness Therapy-Fatigue (FACIT-F) score from the baseline at 52 weeks after the initial administration;
   vi. an improvement in 36-Item Short Form Health Survey questionnaire version 2 (SF-36v2) physical and mental composite score from the baseline at 52 weeks after the initial administration; and
   vii. an improvement in Patient-reported Sjögren's disease diary from the baseline at 52 weeks after the initial administration.
[A68] The method of A1 to A67, wherein the subject or each of the subjects does not have another active autoimmune rheumatic disease.
[A69] The method of A1 to A68, wherein the subject or each of the subjects has not received a B-cell depleting therapy.
[A70] The method of A69, wherein the subject or each of subjects has not received a B-cell depleting therapy within 12 months prior to the administration or if received, the B-cell count is less than the lower limit of normal or baseline value prior to the receipt of previous B cell-depleting therapy (whichever is lower).
[A71] The method of A1 to A70, wherein the subject or each of the subjects has not previously treated with one or more of the following within at least 6 months prior to the administration: belimumab, abatacept, anti-tumor necrosis factor alpha biologic agents, immunoglobulins, plasmapheresis; intravenous (IV) or oral cyclophosphamide, and mycophenolate mofetil, IV or oral cyclosporine A or any other immunosuppressants.
[A72] The method of A1 to A71, wherein the subject or each of the subjects does not exhibit the following laboratory values at screening:
   i. Hemoglobin levels <8.0 g/dL;
   ii. White blood cells (WBC) count <2.0×103/μL;
   iii. Platelet count <100×103/μL;
   iv. Absolute neutrophil count (ANC)<0.8×103/μL; and
   v. Estimated glomerular filtration rate (eGFR) calculated using the 2021 Chronic Kidney Disease-Epidemiology Collaboration (CKD-EPI) serum creatinine eGFR formula <45 mL/min/1.73 m2.
[A73] The method of A1 to A72, wherein the subject or each of the subjects does not exhibit active viral, bacterial, or other infections requiring systemic treatment at the time of screening or randomization, or history of recurrent clinically significant infection.
[A74] The method of A1 to A73, wherein the subject or each of the subjects does not have history of a previous hypersensitivity or severe allergic reaction with generalized urticaria, angioedema, or anaphylaxis to any of the ingredients of the anti-APRIL antibody SC injection formulation.
[A75] The method of A1 to A74, wherein the subject or each of the subjects has not had a history of major organ, hematopoietic stem cell, or bone marrow transplant.
[A76] The method of A1 to A75, wherein the subject or each of the subjects does not take a medication known to cause dry mouth/eyes as a regular and major side effect, and which have not been on a stable dose for at least 30 days prior to the administration, or any anticipated change in the treatment regimen during the course of the administration.
[A77] The method of A1 to A76, wherein the subject or each of the subjects does not use a topical ocular prescription medication (excluding artificial tears, gels, lubricants) that have not been on a stable dose for at least 90 days prior to administration, or any anticipated change in the treatment regimen during the course of the administration.
[A78] The method of A1 to A77, wherein the subject or each of the subjects does not have a history of primary or secondary immunodeficiency, including a positive human immunodeficiency virus (HIV) test result.
[A79] The method of A1 to A78, wherein the subject or each of the subjects has not a history of malignancy of any organ system (other than localized basal cell carcinoma of the skin or in situ cervical cancer), treated or untreated, within the past 5 years, regardless of whether there is evidence of local recurrence or metastases.
[A80] The method of A1 to A79, wherein the subject or each of the subjects does not have sarcoidosis.
[A81] The method of A1 to A80, wherein the subject or each of the subjects has not have a history of fibromyalgia.
[A82] The method of A1 to A81, wherein the subject or each of the subjects does not have uncontrolled type 2 diabetes, as evidenced by a screening hemoglobin A1c (HbA1c) value >8%.
[A83] The method of A1 to A82, wherein the subject or each of the subjects has not a surgical, medical (eg, uncontrolled hypertension, heart failure, cerebrovascular accident), psychiatric or additional physical condition that interferes with the administration.
[A84] The method of A1 to A83, wherein the subject or each of the subjects does not have a positive serology for hepatitis B surface antigen (HBsAg).
[A85] The method of A1 to A84, wherein the subject or each of the subjects does not have hepatitis C.
[A86] The method of A1 to A85, wherein the subject or each of the subjects does not have positive hepatitis C antibody and hepatitis C virus (HCV)-ribonucleic acid (RNA) at the administration or prior to administration except if the subject or each of the subjects has chronic hepatitis C, but has completed HCV anti-viral treatment and is HCV-RNA negative at least 12 weeks after the anti-viral treatment before the administration.
[A87] The method of A1 to A86, wherein the subject or each of the subjects does not have active tuberculosis infection.
[A88] The method of A1 to A87, wherein the subject or each of the subjects is not pregnant or nursing (lactating).
[A89] The method of A1 to A88, wherein the subject or each of the subjects after the administration exhibits mean AUCtau of about 4000 day*μg/ml to about 6000 day*μg/ml, e.g., 4000 day*μg/ml, 4100 day*μg/ml, 4200 day*μg/ml, 4300 day*μg/ml, 4400 day*μg/ml, 4500 day*μg/ml, 4600 day*μg/ml, 4700 day*μg/ml, 4800 day*μg/ml, 4900 day*μg/ml, 5000 day*μg/ml, 5100 day*μg/ml, 5200 day*μg/ml, 5300 day*μg/ml, 5400 day*μg/ml, 5500 day*μg/ml, 5600 day*μg/ml, 5700 day*μg/ml, 5800 day*μg/ml, 5900 day*μg/ml, or 6000 day*μg/ml.
[A90] The method of A1 to A88, wherein the subject or each of the subjects after the administration exhibits mean (SD) AUCtau of about 4830 (2780) day*μg/mL.
[A91] The method of A1 to A90, wherein the subject or each of the subjects after the administration exhibits mean (SD) Cmax of about 100 μg/ml to about 300 μg/ml, e.g., 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, 290 μg/ml, or 300 μg/ml.
[A92] The method of A1 to A90, wherein the subject or each of the subjects after the administration exhibits mean (SD) Cmax of about 194(100) g/ml.
[A93] The method of A1 to A92, wherein the subject or each of the subjects after the administration exhibits mean Ctrough of about 100 μg/ml to about 200 μg/ml, e.g., 110 μg/ml, 120 μg/ml, 130 μg/ml, 140 μg/ml, 150 μg/ml, 160 μg/ml, 170 μg/ml, 180 μg/ml, 190 μg/ml or 200 g/ml.
[A94] The method of A1 to A92, wherein the subject or each of the subjects after the administration exhibits mean (SD) Ctrough of about 141 (94) g/mL.
[A95] The method of A1 to A88, wherein the subject or each of the subjects after the administration exhibits mean (SD) Ctrough of about 141 (94) g/mL, mean (SD) Cmax of about 194(100) g/ml, mean (SD) AUCtau of about 4830 (2780) day*μg/mL, or any combination thereof.
[A96] The method of A1 to A95, wherein the subject or each of the subjects does not have a mixed connective-tissue disease or Sjögren overlap syndrome.
[A97]A pharmaceutical composition comprising: (a) an anti-APRIL antibody and
(b) one or more ingredients comprising L-arginine, L-glutamic acid, L-histidine, L-histidine monohydrochloride, polysorbate 80, and/or sorbitol.
[A98]A pharmaceutical composition comprising: (a) an anti-APRIL antibody and
(b) one or more ingredients comprising L-arginine, L-glutamic acid, L-histidine, polysorbate 80, and/or sorbitol.
[A99] The pharmaceutical composition of any one of A97-98, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8.
[A100] The pharmaceutical composition of any one of A97-99, wherein the VH comprises the amino acid sequence as set forth in SEQ ID NO: 1.
[A10l] The pharmaceutical composition of any one of A97-100, wherein the VL comprises the amino acid sequence as set forth in SEQ ID NO: 2.
[A102] The pharmaceutical composition of any one of A97-101, wherein the anti-APRIL antibody further comprises a constant region linked to the VH and or VL.
[A103] The pharmaceutical composition of any one of any one of A97-102, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A104] The pharmaceutical composition of any one of A97 or A98, wherein the anti-APRIL antibody comprises zigakibart/BION-1301.
[A105] The pharmaceutical composition of any one of A97-104, which is a liquid formulation.
[A106] The pharmaceutical composition of any one of A97-105, which is a liquid formulation for subcutaneous injection.
[A107] The pharmaceutical composition of any one of A97-106, which is contained within a syringe.
[A108] The pharmaceutical composition of any one of A97-107, which is contained within an autoinjector.
[A109] The pharmaceutical composition of any one of A97-108, wherein the pH of the pharmaceutical composition is between about 5 and about 7, e.g., about 5.0, about 5.25, about 5.5, about 5.75, about 6.0, about 6.25, about 6.5, about 6.75, or about 7.0.
[A110] The pharmaceutical composition of A109, wherein the pH is about 6.2.
[A111] The pharmaceutical composition of any one of A97-110, wherein the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL.
[A112] The pharmaceutical composition of any one of A97-111, wherein the total volume of the composition is about 2 mL.
[A113] The pharmaceutical composition of any one of A97-112, which comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol.
[A114] The pharmaceutical composition of A113, which further comprises water for injection.
[A115] The pharmaceutical composition of any one of claims A111-114, wherein the pH of the composition is about 6.2.
[A116] The pharmaceutical composition of any one of A97-115, wherein the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol.
[A117] The pharmaceutical composition of any one of A97-116, wherein the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2.
[A118] The pharmaceutical composition of any one of A97-117, wherein the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol.
[A119] The pharmaceutical composition of any one of A97-118, further comprising a trace amount of a chloride ion.
[A120] The pharmaceutical composition of any one of A97-119, wherein the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg.
[A121] The pharmaceutical composition of any one of A97-120, wherein the composition comprises about 400 mg of the anti-APRIL antibody.
[A122] The pharmaceutical composition of any one of A97-121, further comprising an overfill.
[A123] The pharmaceutical composition of A122, wherein the overfill comprises about 0.0125 mL to about 0.5 mL.
[A124] The pharmaceutical composition of any one of A122-123, wherein the overfill comprises about 0.025 mL to about 0.5 mL, about 0.025 to about 0.05 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL.
[A125] The pharmaceutical composition of any one of A122-124, wherein the overfill comprises about 0.0125 mL, about 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.
[A126]A pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A127]A pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.
[A128] The pharmaceutical composition of A126 or 127, wherein the anti-APRIL antibody in the composition is about 400 mg.
[A129] The pharmaceutical composition of any one of A97 to 128, wherein the anti-APRIL antibody comprises Sibeprenlimab or an antigen binding portion thereof.
[A130]A method of treating a disease or disorder associated with APRIL, e.g., Sjögren's disease, in a subject in need thereof comprising administering to the subject the pharmaceutical composition of any one of A97 to 129.
[A131]A method of producing a pharmaceutical composition comprising admixing one or more ingredients in the pharmaceutical composition of any one of A97 to 129.

### DETAILED DESCRIPTION

Disclosed herein are methods of treating or presenting disorders and conditions associated with APRIL, e.g., Sjögren's disease.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical aspect, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some aspects, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In certain aspects, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof, amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” e.g., Sjögren's disease, means that a subject (e.g., a human) who has a disorder, e.g., Sjögren's disease, and/or experiences a symptom of a disorder, e.g., Sjögren's disease, will, in an aspect, suffer less a severe symptom and/or recover faster when an antibody molecule is administered than if the antibody molecule were never administered. Assays, urine tests, blood tests, iothalamate clearance tests, or kidney imaging (e.g., ultrasound, X-rays, or cystoscopy), for example, can be used to monitor treatment in a patient, or to detect the presence, e.g., decreased presence (or absence), of a symptom of Sjögren's disease, after treatment of Sjögren's disease in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., Sjögren's disease. In some aspects, treatment is of a subject who does not exhibit certain signs of a disorder, e.g., Sjögren's disease, and/or of a subject who exhibits only early signs of a disorder. In some aspects, treatment is of a subject who exhibits one or more established signs of a disorder, e.g., Sjögren's disease. In some aspects, treatment is of a subject diagnosed as suffering from a disorder, e.g., Sjögren's disease.

As used herein, the term “prevent,” a disorder, e.g., Sjögren's disease, means that a subject (e.g., a human) is less likely to have the disorder, e.g., Sjögren's disease, if the subject receives the antibody molecule.

As used herein, the term “baseline” as used herein means measurements or data collected or present at the beginning of a patient's evaluation or treatment. This baseline is important because it serves as a reference point for monitoring the progression of the disease, assessing the effectiveness of treatments, and identifying any changes over time. Baseline in Sjögren's disease can include (i) symptoms assessment (e.g., evaluation of common symptoms like dry eyes, dry mouth (xerostomia), fatigue, and joint pain that are often measured using standardized questionnaires or scales (e.g., the Sjögren's Syndrome Patient Reported Index or ESSDAI), (ii) clinical tests such as: (1) Schirmer's test for tear production (measuring dryness of the eyes), (2) Sialography or salivary gland scintigraphy to assess salivary gland function, (3) blood tests for markers like anti-SSA and anti-SSB antibodies, which are commonly elevated in Sjögren's, (iii) organ involvement (e.g., evaluation of other potential organ involvement, such as lung, kidney, or nervous system involvement, which is important in systemic forms of Sjögren's), or (iv) inflammatory markers (e.g, levels of acute-phase reactants (e.g., C-reactive protein (CRP), erythrocyte sedimentation rate (ESR).

Various aspects of the compositions and methods herein are described in further detail below. Additional definitions are set out throughout the specification.

### Method of Treatment

The present disclosure provides a method of treating a Sjögren's disease in a subject in need thereof comprising administering an anti-APRIL antibody molecule disclosed herein.

Sjögren's disease, or Sjögren's syndrome, is an autoimmune disorder characterized, in some aspects, by dry eyes and dry mouth, stemming from the immune system's attack on the glands that produce moisture. It can be classified as either primary, occurring on its own, or secondary, where it accompanies other autoimmune conditions like rheumatoid arthritis or lupus. The exact cause of Sjögren's remains unclear, but genetic, environmental, and hormonal factors may contribute to its development.

In addition to the hallmark symptoms of xerophthalmia (dry eyes) and xerostomia (dry mouth), Sjögren's can affect various body systems, leading to fatigue, joint pain, and even complications in organs such as the kidneys and liver. Patients may also experience swollen salivary glands, increased dental cavities, and difficulties with swallowing. The disease can significantly impact quality of life, making everyday activities challenging.

Diagnosis typically involves a combination of clinical evaluation, blood tests for specific autoantibodies, and possibly imaging or biopsy of the salivary glands. While there is no cure for Sjögren's disease, treatment focuses on managing symptoms and may include artificial tears for dry eyes, saliva substitutes for dry mouth, and medications to address systemic symptoms. Regular monitoring and a multidisciplinary approach are crucial for managing this complex condition and improving patients' quality of life.

As used herein, the term “subject” is intended to include human and non-human animals. In some aspects, the subject is a human subject, e.g., a human patient having a disorder described herein (e.g., Sjögren's disease), or at risk of having a disorder described herein (e.g., Sjögren's disease). The term “non-human animals” includes mammals and non-mammals, such as non-human primates. The methods and compositions described herein are suitable for treating human patients a disorder described herein (e.g., Sjögren's disease). Patients having a disorder described herein (e.g., Sjögren's disease) include those who have developed a disorder described herein (e.g., Sjögren's disease) but are (at least temporarily) asymptomatic, patients who have exhibited a symptom of a disorder described herein (e.g., Sjögren's disease), or patients having a disorder related to or associated with a disorder described herein (e.g., Sjögren's disease).

In some aspects, the disclosure provides a method of treating Sjögren's disease in a subject in need thereof comprising administering to the subject an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

In some aspects, the disclosure provides a method of treating Sjögren's disease in a population of subjects in need thereof comprising administering to the subjects an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain (VL) CDR1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

In some aspects, an antibody molecule useful for the present disclosure comprises a VH and a VL, wherein the VH comprises the amino acid sequence as set forth in SEQ ID NO: 1. In some aspects, an antibody molecule useful for the present disclosure comprises a VH and a VL, wherein the VL comprises the amino acid sequence as set forth in SEQ ID NO: 2. In some aspects, the antibody molecule further comprises a constant region linked to the VH and or VL.

In some aspects, the present method comprises a method of treating Sjögren's disease in a subject in need thereof comprising administering to the subject an APRIL inhibitor. In some aspects, the APRIL inhibitor comprises an anti-APRIL antibody, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the present method comprises a method of treating Sjögren's disease in a population of subjects in need thereof comprising administering to the subjects an APRIL inhibitor, which comprises an anti-APRIL antibody, wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, one or more of the anti-APRIL antibody useful for the present method (e.g., 2419-1406) are disclosed in WO2017091683A1, published Jun. 1, 2017, which is incorporated herein by reference in its entirety.

In some aspects, the present method comprises a method of treating Sjögren's disease in a subject in need thereof comprising administering to the subject an anti-APRIL antibody, wherein the anti-APRIL antibody comprises Sibeprenlimab or Zigakibart/BION-1301.

In some aspects, the present method provides a method of treating Sjögren's disease in a population of subjects in need thereof comprising administering to the subjects an anti-APRIL antibody, wherein the anti-APRIL antibody comprises Sibeprenlimab or Zigakibart/BION-1301.

In some aspects, the anti-APRIL antibody useful for the present method is disclosed in WO2010/100056, WO2015/034364, WO2016/110587, WO2021/243298 and WO2024/105549, each of which are incorporated herein by reference in their entireties.

In some aspects, the present method comprises a method of treating Sjögren's disease in a subject in need thereof comprising administering to the subject an APRIL inhibitor, which is a fusion protein. In some aspects, an APRIL inhibitor useful for the present method comprises a TACI-fusion protein. In some aspects, an APRIL inhibitor useful for the present method comprises Atacicept. In some aspects, an APRIL inhibitor useful for the present method comprises Povetacicept. In some aspects, the TACI-fusion protein useful for the present disclosure is disclosed in WO2002/094852, WO2021/226551, WO2022/236335, WO2024/123675, or WO2024/077018, which is incorporated herein by reference in its entirety.

In some aspects, the subject or each of the subjects for the present methods has a range of ages between 18 years old and 75 years old.

In some aspects, the methods in a subject can improve the subject's symptoms and disease from the baseline. In some aspects, the methods can improve symptoms associated with Sjögren's syndrome. In some aspects, the present methods can improve common symptoms like dry eyes, dry mouth (xerostomia), fatigue, and joint pain that are often measured using standardized questionnaires or scales (e.g., the Sjögren's Syndrome Patient Reported Index or ESSDAI). In some aspects, the present methods can improve the Sjögren's syndrome that can be measured by clinical tests such as: (1) Schirmer's test for tear production (measuring dryness of the eyes), (2) Sialography or salivary gland scintigraphy to assess salivary gland function, (3) blood tests for markers like anti-SSA and anti-SSB antibodies. In some aspects, the present methods can improve organ involvement in Sjögren's syndrome, such as lung, kidney, or nervous system involvement. In some aspects, the present methods improve (i.e., reduce) inflammatory markers in a subject with Sjögren's syndrome (e.g, levels of acute-phase reactants (e.g., C-reactive protein (CRP), erythrocyte sedimentation rate (ESR).

In some aspects, the subject or each of the subjects exhibits after the administration, exhibits one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

In some aspects, after the administration, the subject or each of the subjects exhibits one or more of the following:
a. a decrease in individual ESSDAI domains from the baseline at 52 weeks after the initial administration;
b. an increase in the salivary flow rate and/or tear flow rate, e.g., 0.1 mL/min or higher from the baseline at 52 weeks after the initial administration;
c. a decrease in Physician's global assessment (PhGA) score of disease activity from the baseline at 52 weeks after the initial administration;
d. a decrease in Patient's global assessment (PaGA) score of participant outcomes from the baseline at 52 weeks after the initial administration;
e. a decrease in Functional Assessment of Chronic Illness Therapy-Fatigue (FACIT-F) score from the baseline at 52 weeks after the initial administration;
f. an improvement in 36-Item Short Form Health Survey questionnaire version 2 (SF-36v2) physical and mental composite score from the baseline at 52 weeks after the initial administration; and
g. an improvement in Patient-reported Sjögren's disease diary from the baseline at 52 weeks after the initial administration.

### ESSDAI

In some aspects, the subject or each of the subjects can be treated based on a European League against Rheumatism (EULAR) Sjögren's syndrome disease activity index (ESSDAI) score. The ESSDAI is one of the tools used to assess disease activity in patients with Sjögren's syndrome. The ESSDAI evaluates various organ systems affected by Sjögren's syndrome, including: (1) constitutional (fatigue, fever, weight loss), (2) musculoskeletal (arthritis, myalgia), (3) mucosal (dryness in eyes/mouth), (4) glandular (swelling of salivary glands), (5) pulmonary (lung involvement), (6) renal (kidney involvement), (7) neurological (neuropathy, cognitive dysfunction), (8) hematological (anemia, leukopenia), and (9) lymphoproliferative (lymphoma risk). Each domain has a specific score ranging from 0 to 3, with higher scores indicating greater disease activity. The overall ESSDAI score is the sum of the scores from each domain, resulting in a total score that reflects the overall disease activity level. A user guide for ESSDAI can be found at Seror R et al., EULAR Sjögren's syndrome disease activity index (ESSDAI): a user guide. RMD Open. 2015 Feb. 20; 1(1):e000022. doi: 10.1136/rmdopen-2014-000022.

ESSDAI scores can be interpreted as follows:
0-5: Low disease activity
6-13: Moderate disease activity
14 and above: High disease activity

In some aspects, the subject or each of the subjects has a baseline ESSDAI score equal to or higher than 4, 5, 6, 7, 8, 9, 10, 11, 12, 13, 14, 15, 16, 17, 18, 19, 20 21, 22, 23, 24, 25, 26, or 27. In some aspects, the subject or each of the subjects has a baseline ESSDAI score equal to or higher than 4 and equal to or less than 123, 100, 90, 80, 70, 60, 50, 40, 30, 20, 15, 14, 13, 12, 11, or 10. In some aspects, the subject or each of the subjects has a baseline ESSDAI score equal to or higher than 5. In some aspects, the subject or each of the subjects has a baseline ESSDAI score equal to or higher than 4. In some aspects, the subject or each of the subjects has a baseline ESSDAI score equal to or higher than 6. In some aspects, the subject or each of the subjects has a baseline ESSDAI score equal to or higher than 7. In some aspects, the subject or each of the subjects has a baseline ESSDAI score equal to or higher than 8. In some aspects, the subject or each of the subjects has a baseline ESSDAI score equal to or higher than 9. In some aspects, the subject or each of the subjects has a baseline ESSDAI score equal to or higher than 10.

In some aspects, the subject or each of the subjects for the present methods has a baseline ESSDAI score between about 5 and about 27, e.g., about 5 and about 27, about 6 and about 27, about 7 and about 27, about 8 and about 27, about 9 and about 27, or about 10 and about 27. In some aspects, the subject or each of the subjects has a baseline ESSDAI score equal to or higher than 10. In some aspects, the subject or each of the subjects has a baseline ESSDAI score equal to or higher than 15. In some aspects, the subject or each of the subjects has a baseline ESSDAI score equal to or higher than 11. In some aspects, the subject or each of the subjects has a baseline ESSDAI score equal to or higher than 12. In some aspects, the subject or each of the subjects has a baseline ESSDAI score equal to or higher than 13. In some aspects, the subject or each of the subjects has a baseline ESSDAI score equal to or higher than 14.

In some aspects, the subject or each of the subjects for the present methods has a baseline ESSDAI score between about 10 and about 27, about 10, about 11, about 12, about 13, about 14, about 15, about 16, about 17, about 18, about 19, about 20, about 21, about 22, about 23, about 24, about 25, about 26, or about 27. In some aspects, the subject or each of the subjects for the present methods has a baseline ESSDAI score between about 20 and about 27, e.g., about 20, about 21, about 22, about 23, about 24, about 25, about 26, or about 27.

In some aspects, the subject or each of the subjects after the administration exhibits a decrease in EULAR Sjögren's syndrome disease activity index (ESSDAI) by 1 point, 2 points, or 3 points from the baseline at 28 weeks after the initial administration. In some aspects, the subject or each of the subjects after the administration exhibits a decrease in EULAR Sjögren's syndrome disease activity index (ESSDAI) by 3 points from the baseline at 28 weeks after the initial administration. For example the subject's administered an anti-APRIL antibody discosed herein can have a decreased ESSDAI score from 15 to 12 or 11. In some aspects, the subject or each of the subjects exhibits a decrease in EULAR Sjögren's Syndrome Patient Reported Index ESSPRI by 1 point from the baseline at 28 weeks after the initial administration. In some aspects, the subject or each of the subjects after the administration exhibits a decrease in EULAR Sjögren's syndrome disease activity index (ESSDAI) by 2 points from the baseline at 28 weeks after the initial administration. In some aspects, the subject or each of the subjects after the administration exhibits a decrease in EULAR Sjögren's syndrome disease activity index (ESSDAI) by 4 points from the baseline at 28 weeks after the initial administration. In some aspects, the subject or each of the subjects after the administration exhibits a decrease in EULAR Sjögren's syndrome disease activity index (ESSDAI) by 5 points from the baseline at 28 weeks after the initial administration. In some aspects, the subject or each of the subjects after the administration exhibits a decrease in EULAR Sjögren's syndrome disease activity index (ESSDAI) by 6-10, e.g., 6, 7, 8, 9, or 10, points from the baseline at 28 weeks after the initial administration.

In some aspects, after the administration, the subject or each of the subjects exhibits a decrease in EULAR Sjögren's syndrome disease activity index (ESSDAI) by 1 point, 2 points, or 3 points from the baseline at 52 weeks after the initial administration. In some aspects, after the administration, the subject or each of the subjects exhibits a decrease in EULAR Sjögren's syndrome disease activity index (ESSDAI) by 3-10 points, e.g., 3, 4, 5, 6, 7, 8, 9, or 10 from the baseline at 52 weeks after the initial administration. In some aspects, after the administration, the subject or each of the subjects exhibits a decrease in EULAR Sjögren's syndrome disease activity index (ESSDAI) by 1 or 2 points from the baseline at 52 weeks after the initial administration. In some aspects, after the administration, the subject or each of the subjects exhibits a decrease in EULAR Sjögren's Syndrome Patient Reported Index ESSPRI by 1 point from the baseline at 52 weeks after the initial administration.

### Salivary Flow Rate

In some aspects, the subject or each of the subjects for the present methods can be administered based on a stimulated whole salivary flow rate. Stimulated whole salivary flow rate can be an important diagnostic measure in Sjögren's syndrome. This measurement assesses the amount of saliva produced over a specific period (usually 15 minutes) after stimulation, typically using a sour stimulus like lemon or citric acid. Saliva is collected during this stimulation period, and the volume is measured in milliliters (mL). The flow rate can indicate the severity of gland involvement. Lower rates suggest more severe dysfunction, which can guide treatment decisions. Normal stimulated whole salivary flow rate is generally considered to be above 1.0 mL/min. Values below this threshold may indicate dysfunction and warrant further evaluation for conditions like Sjögren's syndrome.

In some aspects, the subject or each of the subjects has a stimulated whole salivary flow rate less than 1.0 mL/min, prior to the administration. In some aspects, the subject or each of the subjects has a stimulated whole salivary flow rate of equal to or higher than 0.05 mL/min and below 1.0 mL/min. In some aspects, the subject or each of the subjects has a stimulated whole salivary flow rate of equal to or higher than 0.06 mL/min and below 1.0 mL/min. In some aspects, the subject or each of the subjects has a stimulated whole salivary flow rate of equal to or higher than 0.07 mL/min and below 1.0 mL/min. In some aspects, the subject or each of the subjects has a stimulated whole salivary flow rate of equal to or higher than 0.08 mL/min and below 1.0 mL/min. In some aspects, the subject or each of the subjects has a stimulated whole salivary flow rate of equal to or higher than 0.09 mL/min and below 1.0 mL/min. In some aspects, the subject or each of the subjects has a stimulated whole salivary flow rate of equal to or higher than 0.1 mL/min and below 1.0 mL/min. In some aspects, the subject or each of the subjects has a stimulated whole salivary flow rate of equal to or higher than 0.04 mL/min and below 1.0 mL/min.

In some aspects, the subject or each of the subjects exhibits an increase in the salivary flow rate, e.g., 0.1, 0.2, 0.3, 0.4, 0.5, 0.6, 0.7, 0.8, 0.9, or 1.0 mL/min or higher (up to 2 mL/min) from the baseline at 28 weeks after the initial administration. In some aspects, the subject or each of the subjects exhibits an increase in the salivary flow rate by at least 0.5 mL/min from the baseline at 28 weeks after the initial administration. In some aspects, the subject or each of the subjects exhibits an increase in the salivary flow rate by at least 0.6 mL/min from the baseline at 28 weeks after the initial administration. In some aspects, the subject or each of the subjects exhibits an increase in the salivary flow rate by at least 0.7 mL/min from the baseline at 28 weeks after the initial administration. In some aspects, the subject or each of the subjects exhibits an increase in the salivary flow rate by at least 0.8 mL/min from the baseline at 28 weeks after the initial administration. In some aspects, the subject or each of the subjects exhibits an increase in the salivary flow rate by at least 0.9 mL/min from the baseline at 28 weeks after the initial administration. In some aspects, the subject or each of the subjects exhibits an increase in the salivary flow rate by at least 1.0 mL/min from the baseline at 28 weeks after the initial administration. In some aspects, the subject or each of the subjects exhibits an increase in the salivary flow rate by at least 1.5 mL/min from the baseline at 28 weeks after the initial administration. In some aspects, the subject of each of the subjects exhibits a resting salivary flow rate between 0.1 mL/min and 0.4 mL/min, after the administration. In some aspects, the subject of each of the subjects exhibits a stimulated salivary flow rate between 1 mL/min and 2 mL/min, after the administration.

### Tear Flow Rate

In Sjögren's disease, where there is typically dry eye (keratoconjunctivitis sicca), tear production is often decreased. A result of <5 mm of wetting after 5 minutes is considered abnormal and may be indicative of dry eye syndrome or Sjögren's disease. A tear flow rate can be measured by a Schirmer test. For the test, a small strip of filter paper is placed inside the lower eyelid, and the patient is asked to keep their eyes closed (without blinking excessively) for 5 minutes. The amount of wetting or moisture on the paper is then measured in millimeters. To measure stimulated tear production, tear flow can also be assessed using a phenol red thread test, where a small red thread is placed in the lower conjunctival sac. The amount of red staining over a certain period (e.g., 15 seconds) helps determine the tear flow rate.

In some aspects, the subject or each of the subjects for the present methods can be administered based on a stimulated tear flow rate. In some aspects, the subject or each of the subjects for the present methods has a baseline tear flow rate of less than 5 mm of wetting after 5 minutes. In some aspects, the subject or each of the subjects for the present methods has a baseline tear flow rate of less than 4 mm of wetting after 5 minutes. In some aspects, the subject or each of the subjects for the present methods has a baseline tear flow rate of less than 3 mm of wetting after 5 minutes. In some aspects, the subject or each of the subjects for the present methods has a baseline tear flow rate of less than 2 mm of wetting after 5 minutes. In some aspects, the subject or each of the subjects for the present methods has a baseline tear flow rate of less than 1 mm of wetting after 5 minutes.

In some aspects, the subject or each of the subjects for the present methods exhibits an increased tear flow rate by at least 1 mm, e.g., 1 mm to 10 mm, of wetting after 5 minutes, by an unstimulated Schirmer test, after the administration, at 28 or 52 weeks. In some aspects, the subject or each of the subjects for the present methods exhibits an increased tear flow rate by at least 2 mm of wetting after 5 minutes, by an unstimulated Schirmer test, after the administration, at 28 or 52 weeks. In some aspects, the subject or each of the subjects for the present methods exhibits an increased tear flow rate by at least 3 mm of wetting after 5 minutes, by an unstimulated Schirmer test, after the administration, at 28 or 52 weeks. In some aspects, the subject or each of the subjects for the present methods exhibits an increased tear flow rate by at least 4 mm of wetting after 5 minutes, by an unstimulated Schirmer test, after the administration, at 28 or 52 weeks. In some aspects, the subject or each of the subjects for the present methods exhibits an increased tear flow rate by at least 5 mm of wetting after 5 minutes, by an unstimulated Schirmer test, after the administration, at 28 or 52 weeks. In some aspects, the subject or each of the subjects for the present methods exhibits an increased tear flow rate by at least 6 mm of wetting after 5 minutes, by an unstimulated Schirmer test, after the administration, at 28 or 52 weeks. In some aspects, the subject or each of the subjects for the present methods exhibits an increased tear flow rate by at least 7 mm of wetting after 5 minutes, by an unstimulated Schirmer test, after the administration, at 28 or 52 weeks. In some aspects, the subject or each of the subjects for the present methods exhibits an increased tear flow rate by at least 8 mm of wetting after 5 minutes, by an unstimulated Schirmer test, after the administration, at 28 or 52 weeks. In some aspects, the subject or each of the subjects for the present methods exhibits an increased tear flow rate by at least 9 mm of wetting after 5 minutes, by an unstimulated Schirmer test, after the administration, at 28 or 52 weeks. In some aspects, the subject or each of the subjects for the present methods exhibits an increased tear flow rate by at least 10 mm of wetting after 5 minutes, by an unstimulated Schirmer test, after the administration, at 28 or 52 weeks.

### Physician's Global Assessment

In some aspects, the subject or each of the subjects exhibits a decrease in Physician's global assessment (PhGA) score of disease activity from the baseline at 28 weeks after the initial administration. The Physician's Global Assessment (PhGA) score is a clinical tool used to evaluate a patient's overall disease activity and health status, particularly in the context of autoimmune diseases like rheumatoid arthritis, systemic lupus erythematosus, and Sjögren's syndrome. The PhGA is designed to provide a standardized way for physicians to assess the overall severity of a patient's condition. It helps in tracking disease progression and evaluating treatment effectiveness. The PhGA is typically rated on a visual analog scale (VAS) ranging from 0 to 10, where: 0 represents “no disease activity” or “very well controlled,” and 10 represents “extremely active disease” or “very poor health,” In some aspects, the subject or each of the subjects maintains low scores (0-3) that indicate low disease activity or well-controlled disease, after the administration. In some aspects, the subject or each of the subjects maintains moderate scores (4-6) that suggest moderate disease activity and may require further evaluation or treatment adjustments. In some aspects, the subject or each of the subjects, after the administration, exhibits less than high scores (7-10) that indicate high disease activity, necessitating prompt intervention or a change in therapeutic strategy. In some aspects, the subject or each of the subjects exhibits a decrease in Physician's global assessment (PhGA) score of disease activity from the baseline at 52 weeks after the initial administration.

### Patient's Global Assessment

In some aspects, the subject or each of the subjects exhibits a decrease in Patient's global assessment (PaGA) score of participant outcomes from the baseline at 28 weeks after the initial administration. The Patient's Global Assessment (PGA) score is a self-reported measure used to evaluate a patient's overall perception of their health status and disease activity. It is commonly used in clinical settings, particularly for chronic conditions like autoimmune diseases, including rheumatoid arthritis and systemic lupus erythematosus. The PGA score helps capture the patient's subjective experience of their health and disease activity. It complements objective clinical assessments, providing a more comprehensive understanding of the patient's condition. The PGA is typically assessed using a visual analog scale (VAS) or a numeric scale: On a VAS, patients mark their level of health on a line, usually ranging from 0 (representing “very well”) to 10 (representing “very poorly” or “extremely active disease”). A numeric scale may also be used, often ranging from 0 to 10, with similar endpoints. Patients complete the PGA questionnaire based on their perception of their health, symptoms, and overall well-being. When filling out the PGA, patients consider various aspects of their condition, including: pain levels, fatigue, functional limitations, emotional well-being, and impact of symptoms on daily activities. In some aspects, the subject or each of the subjects exhibit, after the administration, low scores (0-3) of PGA that indicate the patient feels well or has minimal disease activity. In some aspects, the subject or each of the subjects exhibit, after the administration, moderate scores (4-6) of PGA that suggest a moderate level of disease activity and may warrant further evaluation or treatment adjustments. In some aspects, the subject or each of the subjects, after the administration, exhibit less than high scores (7-10) of PGA that indicate significant disease activity or poor health status, often requiring immediate attention or a change in treatment strategy. In some aspects, the subject or each of the subjects exhbits a decrease in Patient's global assessment (PaGA) score of participant outcomes from the baseline at 52 weeks after the initial administration.

### Functional Assessment of Chronic Illness Therapy-Fatigue (FACIT-F)

In some aspects, the subject or each of the subjects exhbits a decrease in Functional Assessment of Chronic Illness Therapy-Fatigue (FACIT-F) score from the baseline at 28 weeks after the initial administration. The Functional Assessment of Chronic Illness Therapy-Fatigue (FACIT-F) score is a standardized questionnaire designed to measure fatigue and its impact on the quality of life in individuals with chronic illnesses. It is particularly useful in assessing fatigue related to various conditions, including cancer, rheumatoid arthritis, and other chronic diseases. The FACIT-F scale aims to quantify the severity of fatigue and its impact on daily functioning and overall well-being. It helps clinicians understand how fatigue affects patients' lives, which can inform treatment and management strategies. The FACIT-F questionnaire typically consists of 13 items to measure the frequency and severity of fatigue and the impact of fatigue on physical, social, emotional, and functional well-being. Each item is rated on a 5-point Likert scale, where: 0=Not at all, 1=A little bit, 2=Somewhat, 3=Quite a bit, and 4=Very much. The total score is calculated by summing the responses, with higher scores indicating better quality of life and less fatigue. In some aspects, the subject or each of the subjects exhibits a decrease in Functional Assessment of Chronic Illness Therapy-Fatigue (FACIT-F) score from the baseline at 52 weeks after the initial administration.

### SF-36v2 Physical and Mental Composite Score

In some aspects, the subject or each of the subjects exhibits an improvement in 36-Item Short Form Health Survey questionnaire version 2 (SF-36v2) physical and mental composite score from the baseline at 28 weeks after the initial administration. The 36-Item Short Form Health Survey version 2 (SF-36v2) is a widely used tool for assessing health-related quality of life. It measures various dimensions of physical and mental health through a comprehensive questionnaire. The SF-36v2 is particularly valuable in both clinical settings and research for evaluating patient outcomes. The SF-36v2 consists of 36 items that evaluate eight domains of health: (1) physical functioning; (2) role limitations due to physical health problems; (3) role limitations due to emotional problems; (4) energy/fatigue; (5) emotional well-being; (6) social functioning; (7) pain; and (8) general health perceptions. Responses to the questionnaire are scored and transformed into two summary scores: (i) physical composite score (PCS) that reflects overall physical health and mental composite score (MCS) that reflects overall mental health. The PCS provides an overall measure of physical functioning and well-being. It incorporates responses related to physical limitations, pain, and general health. It is derived from the physical functioning, role-physical, bodily pain, and general health domains. Higher scores indicate better physical health and functioning, while lower scores suggest greater physical impairment or limitations. The MCS assesses mental health status and emotional well-being. It reflects how mental health issues may affect daily activities and quality of life. It is derived from the emotional well-being, role-emotional, social functioning, and energy/fatigue domains. Higher scores indicate better mental health and emotional well-being, while lower scores suggest increased emotional distress or mental health challenges. By providing both PCS and MCS, the SF-36v2 offers a holistic view of a patient's health status, capturing both physical and mental aspects of well-being. The scores can be used to track changes over time after the administration of the anti-APRIL antibody, allowing healthcare providers to assess the impact of treatments or interventions on both physical and mental health. In some aspects, the subject or each of the subjects exhibits an improvement in 36-Item Short Form Health Survey questionnaire version 2 (SF-36v2) physical and mental composite score from the baseline at 52 weeks after the initial administration. An example of SF-36 can be found at www.physio-pedia.com/36-Item_Short_Form_Survey_(SF-36) (last checked Dec. 1, 2025). See also Lins L, Carvalho F M. SF-36 total score as a single measure of health-related quality of life: Scoping review. SAGE Open Med. 2016 Oct. 4; 4:2050312116671725. doi: 10.1177/2050312116671725.

### Patient-Reported Sjögren's Disease Diary (PRSDD)

In some aspects, the subject or each of the subjects exhibits an improvement in Patient-reported Sjögren's disease diary from the baseline at 28 weeks after the initial administration. Patient-Reported Sjögren's Disease Diary (PRSDD) is a tool designed to capture daily patient-reported outcomes related to the symptoms and impacts of Sjögren's syndrome. The diary is used to monitor and track disease activity and symptoms, providing both the patient and the healthcare provider with real-time insights into the progression and management of the disease. The PRSDD allows patients to record daily symptoms of Sjögren's syndrome, such as dry mouth, dry eyes, joint pain, fatigue, and other systemic manifestations. This helps in better understanding the severity and fluctuations of symptoms over time. While the specific structure of the diary can vary depending on the version used, typical components often include: symptom assessment (dry mouth, dry eyes, fatigue, joint pain, and other symptoms such as rashes, difficulty swallowing, or issues related to the digestive system, lungs, or kidneys), impact on daily life (functional impact and quality of fife), medication and treatment tracking (current medications and effectiveness of treatment), flare-ups and exacerbations (recording flare-ups). In some aspects, the subject or each of the subjects exhibits an improvement in Patient-reported Sjögren's disease diary from the baseline at 52 weeks after the initial administration.

### Anti-SSA/Ro Antibody

In some aspects, the subject or each of the subjects is seropositive for an anti-Sjögren's-syndrome-related antigen A (anti-SSA/Ro) antibody. Anti-Sjögren's-syndrome-related antigen A (anti-SSA/Ro) antibodies are autoantibodies commonly associated with autoimmune diseases, particularly Sjögren's syndrome (SLE). These antibodies target specific proteins (Ro52 and Ro60) found in the cell nucleus and cytoplasm. They are part of the body's immune response, mistakenly attacking its own tissues.

In Sjögren's syndrome, anti-SSA/Ro antibodies are present in a significant number of patients. Their presence can indicate an increased risk of developing the disease and is often used as a diagnostic marker. In some aspects, a subject with a positive test for anti-SSA/Ro antibodies supports the diagnosis of Sjögren's syndrome, especially in conjunction with other clinical findings, can be administered an anti-APRIL antibody disclosed herein.

In some aspects, the subject or each of the subjects exhibits a decrease in serum anti-drug antibody (ADA). Serum Anti-Drug Antibodies (ADAs) are antibodies produced by the immune system in response to therapeutic drugs, i.e., anti-APRIL antibody described herein. These antibodies can form when the body recognizes the anti-APRIL antibody as a foreign substance and mounts an immune response. The presence of anti-drug antibodies (ADAs) in a patient's serum can affect both the efficacy and safety of the drug.

In some aspects, an ADA is a neutralizing antibody. In some aspects, an ADA is a non-neutralizing antibody. In some aspects, the treatment is terminated if the subject exhibits an increase in serum ADA, or no decrease in serum ADA.

In some aspects, the subject or each of the subjects exhibits an incidence of treatment emergent adverse events (TEAEs) graded by severity, clinical laboratory tests, vital sign measurements, physical examinations, injection site reactions, or any combination thereof. In some aspects, the subject or each of the subjects exhibits reduction of serum IgG. In some aspects, In some aspects, the subject or each of the subjects exhibits reduction in circulating peripheral immune cells. In some aspects, the subject or each of the subjects exhibits reversible reductions in total serum IgA. In some aspects, the subject or each of the subjects exhibits reversible reductions in total serum IgM and IgG. In some aspects, the subject or each of the subjects exhibits reduction in mononuclear cells staining positive for IgA, IgG, and/or IgM in GALT and/or tonsils. In some aspects, the subject or each of the subjects exhibits reduction of serum IgG at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reduction of serum IgG at a level between about 60% to about 95%.

In some aspects, the subject or each of the subjects exhibits reduction in circulating peripheral immune cells at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reversible reductions in total serum IgA at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reversible reductions in total serum IgM and IgG at a level of at least about 10%, about 20%, about 30%, about 40%, about 50%, about 60%, about 70%, about 80%, about 90%, about 95%, or about 99% compared to the serum IgG level prior to the administration. In some aspects, the subject or each of the subjects exhibits reduction in mononuclear cells staining positive for IgA, IgG, and/or IgM in GALT and/or tonsils at a level of at least about 10%, about 20%, about 30%, about 40%, about 50%, about 60%, about 70%, about 80%, about 90%, about 95%, or about 99% compared to the serum IgG level prior to the administration.

In some aspects, after the administration, the subject or each of the subjects exhibits a change in (i) free APRIL concentrations, (ii) immunoglobulins including autoantibodies; (iii) characteristic of Sjögren's B-cell receptor; (iv) repertoire, inflammatory factors, and gene; (v) expression signatures over time; or (vi) any combination thereof.

In some aspects, the subject or each of the subjects for the present methods can be chosen based on an IgG serum concentration. IgG serum concentration is an important parameter in the evaluation and management of Sjögren's syndrome, an autoimmune condition characterized by dry mouth and dry eyes due to the immune system attacking exocrine glands. Many patients with Sjögren's syndrome exhibit elevated levels of IgG in their serum. This elevation is part of the body's immune response to the ongoing inflammation and autoimmune activity characteristic of the disease. High IgG levels can be associated with the presence of other autoantibodies, including anti-SSA/Ro and anti-SSB/La antibodies. In some aspects, the subject or each of the subjects has a serum IgG concentration greater than 900 mg/dL. In some aspects, the subject or each of the subjects has a serum IgG concentration greater than 1000 mg/dL. In some aspects, the subject or each of the subjects has a serum IgG concentration greater than 1100 mg/dL. In some aspects, the subject or each of the subjects has a serum IgG concentration greater than 1200 mg/dL. In some aspects, the subject or each of the subjects has a serum IgG concentration greater than 1300 mg/dL. In some aspects, the subject or each of the subjects has a serum IgG concentration greater than 1400 mg/dL. In some aspects, the subject or each of the subjects has a serum IgG concentration greater than 1500 mg/dL. In some aspects, the subject or each of the subjects has a serum IgG concentration greater than 800 mg/dL. In some aspects, the subject or each of the subjects has a serum IgG concentration greater than 700 mg/dL.

In some aspects, the subject or each of the subjects does not have a mixed connective-tissue disease or Sjögren overlap syndrome. The term “mized connective-tissue disease” (MCTD) as used herein is an autoimmune disorder that features overlapping symptoms of several connective tissue diseases, e.g., Sjögren's syndrome. MCTD is characterized by the presence of high levels of anti-U1 ribonucleoprotein (RNP) antibodies in the blood, along with a combination of clinical features from different connective tissue diseases. The term “Sjögren overlap syndrome” as used herein refers to the presence of Sjögren's syndrome alongside other autoimmune diseases. When Sjögren's syndrome overlaps with other autoimmune conditions, it can complicate both diagnosis and management. Overlap syndrome occurs when a patient with Sjögren's syndrome also exhibits clinical features and laboratory findings indicative of another autoimmune disease, such as systemic lupus erythematosus (SLE), rheumatoid arthritis (RA), scleroderma, or myositis. In some aspects, the subject or each of the subjects does not have a mixed connective-tissue disease or Sjögren overlap syndrome, wherein the subject or each of the subjects does not have another autoimmune rheumatic disease that is active and constitutes the principal illness.

In some aspects, the subject or each of the subjects does not have a mixed connective-tissue disease or Sjögren overlap syndrome, wherein the s ubject or each of the subjects has not received a B-cell depleting therapy within 12 months prior to the administration or if received, the B-cell count is less than the lower limit of normal or baseline value prior to the receipt of previous B cell-depleting therapy (whichever is lower). In some aspects, the subject or each of the subjects does not have a mixed connective-tissue disease or Sjögren disease, wherein the subject or each of the subjects has not previously treated with one or more of the following within at least 6 months prior to the administration: a. belimumab, abatacept, anti-tumor necrosis factor alpha biologic agents, b. immunoglobulins, plasmapheresis; intravenous (IV) or oral cyclophosphamide, and c. mycophenolate mofetil, IV or oral cyclosporine A or any other immunosuppressants.

In some aspects, the subject or each of the subjects does not exhibit the following laboratory values at screening:
a. Hemoglobin levels <8.0 g/dL;
b. White blood cells (WBC) count <2.0×103/μL;
c. Platelet count <100×103/μL;
d. Absolute neutrophil count (ANC)<0.8×103/μL; and
e. Estimated glomerular filtration rate (eGFR) calculated using the 2021 Chronic Kidney Disease-Epidemiology Collaboration (CKD-EPI) serum creatinine eGFR formula <45 mL/min/1.73 m2.

In some aspects, the subject or each of the subjects does not exhibit active viral, bacterial, or other infections requiring systemic treatment at the time of screening or randomization, or history of recurrent clinically significant infection.

In some aspects, the subject or each of the subjects does not have history of a previous hypersensitivity or severe allergic reaction with generalized urticaria, angioedema, or anaphylaxis to any of the ingredients of the anti-APRIL antibody SC injection formulation.

In some aspects, the subject or each of the subjects has not had a history of major organ, hematopoietic stem cell, or bone marrow transplant. In some aspects, the subject or each of the subjects does not take a medication known to cause dry mouth/eyes as a regular and major side effect, and which have not been on a stable dose for at least 30 days prior to the administration, or any anticipated change in the treatment regimen during the course of the administration. In some aspects, the subject or each of the subjects does not use a topical ocular prescription medication (excluding artificial tears, gels, lubricants) that have not been on a stable dose for at least 90 days prior to administration, or any anticipated change in the treatment regimen during the course of the administration. In some aspects, the subject or each of the subjects does not have a history of primary or secondary immunodeficiency, including a positive human immunodeficiency virus (HIV) test result. In some aspects, the subject or each of the subjects has not a history of malignancy of any organ system (other than localized basal cell carcinoma of the skin or in situ cervical cancer), treated or untreated, within the past 5 years, regardless of whether there is evidence of local recurrence or metastases. In some aspects, the subject or each of the subjects does not have sarcoidosis. In some aspects, the subject or each of the subjects has not have a history of fibromyalgia.

In some aspects, the subject or each of the subjects does not have uncontrolled type 2 diabetes, as evidenced by a screening hemoglobin A1c (HbA1c) value >8%. In sme aspects, the subject or each of the subjects has not a surgical, medical (eg, uncontrolled hypertension, heart failure, cerebrovascular accident), psychiatric or additional physical condition that interferes with the administration. In some aspects, the subject or each of the subjects does not have a positive serology for hepatitis B surface antigen (HBsAg). In some aspects, the subject or each of the subjects does not have hepatitis C. In some aspects, the subject or each of the subjects does not have positive hepatitis C antibody and hepatitis C virus (HCV)-ribonucleic acid (RNA) at the administration or prior to administration except if the subject or each of the subjects has chronic hepatitis C, but has completed HCV anti-viral treatment and is HCV-RNA negative at least 12 weeks after the anti-viral treatment before the administration. In some aspects, the subject or each of the subjects does not have active tuberculosis infection. In some aspects, the subject or each of the subjects is not pregnant or nursing (lactating).

### Dosing

In some aspects, the dose of an anti-APRIL antibody or an antigen-binding portion thereof for treating Sjögren's syndrome is administered at a fixed dose between about 10 mg and about 1000 mg, e.g., about 50 mg, about 100 mg, about 200 mg, about 300 mg, about 400 mg, about 500 mg, about 600 mg, about 700 mg, about 800 mg, about 900 mg, or about 1000 mg. In some aspects, the dose of an anti-APRIL antibody or an antigen-binding portion thereof for treating Sjögren's syndrome is administered at a fixed dose of about 400 mg once every four weeks. In some aspects, the dose of an anti-APRIL antibody or an antigen-binding portion thereof for treating Sjögren's syndrome is administered at a fixed dose of about 600 mg once every six weeks. In some aspects, the dose of an anti-APRIL antibody or an antigen-binding portion thereof for treating Sjögren's syndrome is administered at a fixed dose of about 800 mg once every eight weeks. In some aspects, the dose of an anti-APRIL antibody or an antigen-binding portion thereof for treating Sjögren's syndrome is administered at a fixed dose of about 200 mg once every two weeks. In some aspects, the anti-APRIL antibody or the antigen binding portion thereof is administered twice, three times, four times, five times, six times, seven times, eight times, nine times, 10 times, 11 times, 12 times, 13 times, 14 times, or 15 times to treat Sjögren's syndrome.

In some aspects, the anti-APRIL antibody molecule or the antigen binding portion thereof that is to be administered to a subject having Sjögren's syndrome is formulated at a concentration of about 200 mg/ml.

In some aspects, the anti-APRIL antibody or the antigen binding portion thereof is administered to a subject having Sjögren's syndrome for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks. In some aspects, the anti-APRIL antibody or the antigen binding portion thereof is administered to a subject having Sjögren's syndrome for at least 28 weeks. In some aspects, the anti-APRIL antibody or the antigen binding portion thereof is administered to a subject having Sjögren's syndrome for at least 52 weeks.

In some aspects, the anti-APRIL antibody or the antigen binding portion thereof is administered twice to a subject having Sjögren's syndrome at a dose of about 400 mg once every four weeks (i.e., the first dose given in week 0, and the second dose given in week 4). In some aspects, the anti-APRIL antibody or the antigen binding portion thereof is administered three times to a subject having Sjögren's syndrome at a dose of about 400 mg once every four weeks (i.e., the first dose given in week 0, the second dose given in week 4, and the third dose given in week 8). In some aspects, the anti-APRIL antibody or the antigen binding portion thereof is administered four times to a subject having Sjögren's syndrome at a dose of about 400 mg once every four weeks (i.e., the first dose given in week 0, the second dose given in week 4, the third dose given in week 8, and the fourth dose given in week 12). In some aspects, the anti-APRIL antibody or the antigen binding portion thereof is administered five times to a subject having Sjögren's syndrome at a dose of about 400 mg once every four weeks (i.e., the first dose given in week 0, the second dose given in week 4, the third dose given in week 8, the fourth dose given in week 12, and the fifth dose given in week 16). In some aspects, the anti-APRIL antibody or the antigen binding portion thereof is administered six times to a subject having Sjögren's syndrome at a dose of about 400 mg once every four weeks (i.e., the first dose given in week 0, the second dose given in week 4, the third dose given in week 8, the fourth dose given in week 12, the fifth dose given in week 16, and the sixth dose given in week 20). In some aspects, the anti-APRIL antibody or the antigen binding portion thereof is administered seven times to a subject having Sjögren's syndrome at a dose of about 400 mg once every four weeks (i.e., the first dose given in week 0, the second dose given in week 4, the third dose given in week 8, the fourth dose given in week 12, the fifth dose given in week 16, and sixth dose given in week 20, and the seventh dose given in week 24).

In some aspects, the anti-APRIL antibody or the antigen binding portion thereof is administered eight times to a subject having Sjögren's syndrome at a dose of about 400 mg once every four weeks (i.e., the first dose given in week 0, the second dose given in week 4, the third dose given in week 8, the fourth dose given in week 12, the fifth dose given in week 16, and sixth dose given in week 20, the seventh dose given in week 24, and the eighth dose given in week 28). In some aspects, the anti-APRIL antibody or the antigen binding portion thereof is administered nine times to a subject having Sjögren's syndrome at a dose of about 400 mg once every four weeks. In some aspects, the anti-APRIL antibody or the antigen binding portion thereof is administered 10 times to a subject having Sjögren's syndrome at a dose of about 400 mg once every four weeks. In some aspects, the anti-APRIL antibody or the antigen binding portion thereof is administered 11 times to a subject having Sjögren's syndrome at a dose of about 400 mg once every four weeks. In some aspects, the anti-APRIL antibody or the antigen binding portion thereof is administered 12 times to a subject having Sjögren's syndrome at a dose of about 400 mg once every four weeks. In some aspects, the anti-APRIL antibody or the antigen binding portion thereof is administered 13 times to a subject having Sjögren's syndrome at a dose of about 400 mg once every four weeks. In some aspects, the anti-APRIL antibody or the antigen binding portion thereof is administered 14 times to a subject having Sjögren's syndrome at a dose of about 400 mg once every four weeks. In some aspects, the anti-APRIL antibody or the antigen binding portion thereof is administered 15 times to a subject having Sjögren's syndrome at a dose of about 400 mg once every four weeks. In some aspects, the anti-APRIL antibody or the antigen binding portion thereof is administered 16 times to a subject having Sjögren's syndrome at a dose of about 400 mg once every four weeks. In some aspects, the anti-APRIL antibody or the antigen binding portion thereof is administered 17 times to a subject having Sjögren's syndrome at a dose of about 400 mg once every four weeks.

In some aspects, the subject or each of the subjects after the administration exhibits mean AUCtau of about 4000 day*μg/ml to about 6000 day*μg/ml, about 4000 day*μg/ml to about 5500 day*μg/ml, about 4000 day*μg/ml to about 5000 day*μg/ml, about 4000 day*μg/ml to about 4900 day*μg/ml, about 4000 day*μg/ml to about 4800 day*μg/ml, about 4500 day*μg/ml to about 6000 day*μg/ml, about 4500 day*μg/ml to about 5500 day*μg/ml, about 4500 day*μg/ml to about 5000 day*μg/ml, about 4500 day*μg/ml to about 4900 day*μg/ml, or about 4500 day*μg/ml to about 4800 day*μg/ml. In some aspects, the subject or each of the subjects after the administration exhibits mean AUCtau of about 4000 day*μg/ml, about 4100 day*μg/ml, about 4200 day*μg/ml, about 4300 day*μg/ml, about 4400 day*μg/ml, about 4500 day*μg/ml, about 4600 day*μg/ml, about 4700 day*μg/ml, about 4800 day*μg/ml, about 4900 day*μg/ml, about 5000 day*μg/ml, about 5100 day*μg/ml, about 5200 day*μg/ml, about 5300 day*μg/ml, about 5400 day*μg/ml, about 5500 day*μg/ml, about 5600 day*μg/ml, about 5700 day*μg/ml, about 5800 day*μg/ml, about 5900 day*μg/ml, or about 6000 day*μg/ml. In some aspects, the subject or each of the subjects after the administration exhibits mean (SD) AUCtau of about 4830 (2780) day*μg/mL.

In some aspects, the subject or each of the subjects after the administration exhibits mean (SD) Cmax of about 100 μg/ml to about 300 μg/ml, about 100 μg/ml to about 250 μg/ml, about 100 μg/ml to about 200 μg/ml, about 150 μg/ml to about 300 μg/ml, about 150 μg/ml to about 250 μg/ml, or about 150 μg/ml to about 200 μg/ml. In some aspects, the subject or each of the subjects after the administration exhibits mean (SD) Cmax of about 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, 290 μg/ml, or 300 g/ml. In some aspects, the subject or each of the subjects after the administration exhibits mean (SD) Cmax of about 194(100) jg/ml.

In some aspects, the subject or each of the subjects after the administration exhibits mean Ctrough of about 100 μg/ml to about 200 μg/ml, about 100 μg/ml to about 180 μg/ml, about 100 μg/ml to about 160 μg/ml, about 100 μg/ml to about 150 μg/ml, about 120 μg/ml to about 200 μg/ml, about 120 μg/ml to about 180 μg/ml, about 120 μg/ml to about 160 μg/ml, about 120 μg/ml to about 150 μg/ml, about 130 μg/ml to about 200 μg/ml, about 130 μg/ml to about 180 μg/ml, about 130 μg/ml to about 160 μg/ml, or about 130 μg/ml to about 150 μg/ml. In some aspects, the subject or each of the subjects after the administration exhibits mean Ctroagh of about 110 μg/ml, about 120 μg/ml, about 130 μg/ml, about 140 μg/ml, about 150 μg/ml, about 160 μg/ml, about 170 μg/ml, about 180 μg/ml, or about 190 μg/ml or about 200 μg/ml. In some aspects, the subject or each of the subjects after the administration exhibits mean (SD) Ctrough of about 141 (94) jg/mL.

In some aspects, the subject or each of the subjects after the administration exhibits mean (SD) Ctrough of about 141 (94) jg/mL, mean (SD) Cmax of about 194(100) jg/ml, mean (SD) AUCtau of about 4830 (2780) day*μg/mL, or any combination thereof.

The present disclosure also provides a method of treating Sjögren's syndrome in a subject in need thereof or a population of subjects in need thereof comprising administering about 400 mg of sibeprenlimab formulated at a concentration of 200 mg/mL once every four weeks for two times, three times, four times, five times, six times, seven times, or eight times wherein the subject or each of the subjects exhibits after the administration one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

The present disclosure also provides a method of treating Sjögren's syndrome in a subject in need thereof or a population of subjects in need thereof comprising administering about 400 mg of sibeprenlimab formulated at a concentration of 200 mg/mL once every four weeks for two times, three times, four times, five times, six times, seven times, eight times, ten times, 11 times, 12 times, 13 times, 14 times, 15 times, or 16 times, wherein the subject or each subject exhibits one or more of the following:
a. a decrease in individual ESSDAI domains from the baseline at 52 weeks after the initial administration;
b. an increase in the salivary flow rate and/or tear flow rate, e.g., 0.1 mL/min or higher from the baseline at 52 weeks after the initial administration;
c. a decrease in Physician's global assessment (PhGA) score of disease activity from the baseline at 52 weeks after the initial administration;
d. a decrease in Patient's global assessment (PaGA) score of participant outcomes from the baseline at 52 weeks after the initial administration;
e. a decrease in Functional Assessment of Chronic Illness Therapy-Fatigue (FACIT-F) score from the baseline at 52 weeks after the initial administration;
f. an improvement in 36-Item Short Form Health Survey questionnaire version 2 (SF-36v2) physical and mental composite score from the baseline at 52 weeks after the initial administration; and
g. an improvement in Patient-reported Sjögren's disease diary from the baseline at 52 weeks after the initial administration.

### Combination Therapies

The antibody molecules for treating Sjögren's syndrome can be used in combination with other therapies. For example, the combination therapy can include an antibody molecule co-formulated with, and/or co-administered with, one or more additional therapeutic agents, e.g., one or more additional therapeutic agents described herein. In other aspects, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In some aspects, two or more treatments are delivered prophylactically, e.g., before the subject has the disorder or is diagnosed with the disorder. In another aspect, the two or more treatments are delivered after the subject has developed or diagnosed with the disorder. In some aspects, the delivery of one treatment is still occurring when the delivery of the second begins, so that there is overlap. This is sometimes referred to herein as “simultaneous” or “concurrent delivery.” In other aspects, the delivery of one treatment ends before the delivery of the other treatment begins. In some aspects of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some aspects, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some aspects, the present methods further comprise administering to the subject or each of the subjects a standard of care (SOC) therapy. In some aspects, the subject or each of the subjects received a SOC therapy prior to the administration. In some aspects, the subject or each of the subjects received a SOC therapy for at least 30 days, at least 40 days, at least 50 days, at least 60 days, at least 70 days, at least 80 days, at least 90 days, or at least 100 days prior to the administration.

In some aspects, the SOC therapy comprises (i) hydroxychloroquine, (ii) a corticosteroid (e.g., prednisone or prednisolone), and/or (iii) any one of methotrexate, leflunomide, and azathioprine. In some aspects, the SOC therapy comprises (i) hydroxychloroquine, (ii) a corticosteroid (e.g., prednisone or prednisolone), and (iii) methotrexate. In some aspects, the SOC therapy comprises (i) hydroxychloroquine, (ii) a corticosteroid (e.g., prednisone or prednisolone), and (iii) leflunomide. In some aspects, the SOC therapy comprises (i) hydroxychloroquine, (ii) a corticosteroid (e.g., prednisone or prednisolone), and (iii) azathioprine. In some aspects, the SOC therapy comprises hydroxychloroquine, a corticosteroid (e.g., prednisone or prednisolone), methotrexate, leflunomide, azathioprine, or any combination thereof.

In some aspects, the hydroxychloroquine is or has been administered to a subject having Sjögren's syndrome at a dose of ≤400 mg/day. In some aspects, the hydroxychloroquine is or has been administered at a dose of between about 10 mg/day and about 400 mg/day, about 100 mg/day and about 400 mg/day, about 200 mg/day and about 400 mg/day, about 300 mg/day and about 400 mg/day, about 10 mg/day and about 300 mg/day, about 100 mg/day and about 300 mg/day, about 200 mg/day and about 300 mg/day, about 10 mg/day and about 200 mg/day, about 100 mg/day and about 200 mg/day, or about 10 mg/day and about 100 mg/day. In some aspects, the hydroxychloroquine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 150 mg/day, about 200 mg/day, about 250 mg/day, about 300 mg/day, about 350 mg/day, or about 400 mg/day.

In some aspects, the corticosteroid (e.g., prednisone or prednisolone) is or has been administered at a dose of ≤10 mg/day. In some aspects, the corticosteroid (e.g., prednisone or prednisolone) is or has been administered at a dose of between about 1 mg/day and about 10 mg/day, about 2 mg/day and about 10 mg/day, about 4 mg/day and about 10 mg/day, about 6 mg/day and about 10 mg/day, about 8 mg/day and about 10 mg/day, about 2 mg/day and about 8 mg/day, about 4 mg/day and about 8 mg/day, about 6 mg/day and about 8 mg/day, about 2 mg/day and about 6 mg/day, about 4 mg/day and about 6 mg/day. In some aspects, the corticosteroid (e.g., prednisone or prednisolone) is or has been administered at a dose of about 1 mg/day, about 2 mg/day, about 3 mg/day, about 4 mg/day, about 5 mg/day, about 6 mg/day, about 7 mg/day, about 8 mg/day, about 9 mg/day, or about 10 mg/day. In some aspects, the corticosteroid comprises prednisone, prednisolone, cortisone, hydrocortisone, methylprednisolone, hydrocortisone acetate, cortisone acetate, tixocortol pivalate, acetonides, amcinonide, budesonide, desonide, fluocinolone acetonide, fluocinonide, halcinonide, triamcinolone acetonide, Deflazacort (0-isopropylidene derivative), beclometasone, betamethasone, dexamethasone, fluocortolone, halometasone, mometasone, esters, halogenated, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, prednicarbate, or tixocortol pivalate.

In some aspects, the methotrexate is or has been administered at a dose of ≤25 mg/week. In some aspects, the methotrexate is or has been administered at a dose of about 1 mg/week, about 2 mg/week, about 3 mg/week, about 4 mg/week, about 5 mg/week, about 6 mg/week, about 7 mg/week, about 8 mg/week, about 9 mg/week, about 10 mg/week, about 11 mg/week, about 12 mg/week, about 13 mg/week, about 14 mg/week, about 15 mg/week, about 16 mg/week, about 17 mg/week, about 18 mg/week, about 19 mg/week, about 20 mg/week, about 21 mg/week, about 22 mg/week, about 23 mg/week, about 24 mg/week, or about 25 mg/week.

In some aspects, the leflunomide is or has been administered at a dose of ≤20 mg daily. In some aspects, the leflunomide is or has been administered at a dose of about 1 mg daily, about 2 mg daily, about 3 mg daily, about 4 mg daily, about 5 mg daily, about 6 mg daily, about 7 mg daily, about 8 mg daily, about 9 mg daily, about 10 mg daily, about 11 mg daily, about 12 mg daily, about 13 mg daily, about 14 mg daily, about 15 mg daily, about 16 mg daily, about 17 mg daily, about 18 mg daily, about 19 mg daily, about 20 mg daily.

In some aspects, the azathioprine is or has been administered at a dose of ≤150 mg/day. In some aspects, the azathioprine is or has been administered at a dose of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, or about 150 mg/day.

### Anti-APRIL Antibody Molecules

Disclosed herein are use of antibody molecules that bind to APRIL, e.g., an APRIL molecule described herein, for treating Sjögren's disease.

APRIL (A PRoliferation Inducing Ligand), also known as CD256, TNF- and APOL-related Leukocyte Expressed Ligand 2 (TALL-2), or TNF-related Death Ligand 1 (TRDL-1), is a TNF family cytokine encoded by the Tumor Necrosis Factor Ligand Superfamily Member 13 (TNFSF13) gene (also known as APRIL, TALL2, or ZTNF2). APRIL plays a role in a number of biological processes such as signal transduction, regulation of cell proliferation, and IgA class switching (Hahne et al. (1998) J. Exp. Med. 188:1185-1190 (1998); Castigli et al. Proc. Natl. Acad. Sci. U.S.A. 101:3903-3908 (2004)).

APRIL is both functionally and structurally related to BAFF (B Cell Activating Factor F13B) also known as BLyS (B lymphocyte stimulator). Both cytokines are involved in regulating key aspects of innate and adaptive immune functions. Exemplary amino acid and nucleotide sequences of human APRIL are described, e.g., in Hahne et al. J. Exp. Med. 188:1185-1190 (1998); Shu et al. J. Leukoc. Biol. 65:680-683 (1999); Kelly et al. Cancer Res. 60:1021-1027(2000); and Pradet-Balade et al. EMBO J. 21:5711-5720 (2002).

The amino acid sequence of human APRIL (isoform alpha, also referred to as the “canonical” sequence (SEQ ID NO: 11)) is provided as follows.

>huAPRIL
MPASSPFLLAPKGPPGNMGGPVREPALSVALWLSWGAALGAVACA
MALLTQQTELQSLRREVSRLQGTGGPSQNGEGYPWQSLPEQSSDA
LEAWENGERSRKRRAVLTQKQKKQHSVLHLVPINATSKDDSDVTE
VMWQPALRRGRGLQAQGYGVRIQDAGVYLLYSQVLFQDVTFTMGQ
VVSREGQGRQETLFRCIRSMPSHPDRAYNSCYSAGVFHLHQGDIL
SVIIPRARAKLNLSPHGTFLGFVKL

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1, IgG2, IgG3, or IgG4. The antibody molecule can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody molecule can be a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In some aspects, the following definitions are used: AbM definition of CDR1 of the heavy chain variable domain and Kabat definitions for the other CDRs. In some aspects, Kabat definitions are used for all CDRs. In addition, aspects described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., APRIL, or an epitope thereof. With respect to proteins (or protein mimetics), the antigen-binding region typically includes one or more loops (of at least, e.g., four amino acids or amino acid mimics) that form an interface that binds to the antigen, e.g., APRIL. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-APRIL antibody molecule, e.g., an anti-APRIL antibody molecule provided herein, to a target, e.g., APRIL. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In some aspects, a competition binding assay is a quantitative competition assay. In some aspects, a first anti-APRIL antibody molecule is said to compete for binding to the target with a second anti-APRIL antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In some aspects, the antibody can be recombinantly produced, e.g., produced by any suitable phage display or combinatorial methods.

In some aspects, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In some aspects, the non-human antibody is a rodent (mouse or rat antibody). Methods of producing rodent antibodies are known in the art.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In some aspects, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some aspects, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

In some aspects, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In another aspect, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of. Fc receptor binding, antibody glycosylation, the number of cysteine residues, effector cell function, and/or complement function). In some aspects, the antibody molecule has effector function and can fix complement. In another aspect, the antibody molecule does not recruit effector cells or fix complement. In certain aspects, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region.

In some aspects, this disclosure provides a method of making an antibody molecule disclosed herein. The method includes: providing an antigen, e.g., APRIL or a fragment thereof, obtaining an antibody molecule that specifically binds to the antigen; evaluating efficacy of the antibody molecule in modulating activity of the antigen and/or organism expressing the antigen, e.g., APRIL. The method can further include administering the antibody molecule, including a derivative thereof (e.g., a humanized antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid sequences of exemplary antibody molecules are described in Tables 1 and 2, below.

TABLE 1
The amino acid sequences of the heavy chain
variable region (VH) and light chain variable
region (VL) of the exemplary anti-APRIL
antibody (e.g., Sibeprenlimab) are provided
as follows. CDRs are defined according to
the Chothia system are italicized.

| | Amino | SEQ | | | SEQ |
|---|---|---|---|---|---|
| | Acid | ID | | | ID |
| Chain | Sequence | NO | Chothia CDR | | NO |
| VH | QVQLVQSGAE | 1 | HCDR1 | GYTFTDY | 3 |
| | VKKPGASVKV | | HCDR2 | YPLRGS | 4 |
| | SCKASGYTFT | | HCDR3 | HGAYYSN | 5 |
| | DYTIHWVRQA | | | AFDY | |
| | TGQGLEWMGW | | | | |
| | IYPLRGSINY | | | | |
| | AQKFQGRVTM | | | | |
| | TADKSISTVY | | | | |
| | MELSSLRSED | | | | |
| | TAVYFCARHG | | | | |
| | AYYSNAFDYW | | | | |
| | GQGTLVTVSS | | | | |
| VL | EIVMTQSPAT | 2 | LCDR1 | RASESVDNDGIRFLH | 6 |
| | LSVSPGERAT | | LCDR2 | RASTRAT | 7 |
| | LSCRASESVD | | LCDR3 | QQSNKDPYT | 8 |
| | NDGIRFLHWY | | | | |
| | QQKPGQAPRL | | | | |
| | LIYRASTRAT | | | | |
| | GIPARFSGSG | | | | |
| | SRTEFTLTIS | | | | |
| | SLQSEDFAVY | | | | |
| | YCQQSNKDPY | | | | |
| | TFGGGTKVEI | | | | |
| | K | | | | |

TABLE 2
The amino acid sequences of heavy chain (HC)
and light chain (LC) of the exemplary anti-
APRIL antibody (e.g., Sibeprenlimab) are
provided below.

| Feature | Sequence | SEQ ID NO: |
|---|---|---|
| Heavy | QVQLVQSGAEVKKPGASVKV | 9 |
| Chain | SCKASGYTFTDYTIHWVRQA | |
| | TGQGLEWMGWIYPLRGSINY | |
| | AQKFQGRVTMTADKSISTVY | |
| | MELSSLRSEDTAVYFCARHG | |
| | AYYSNAFDYWGQGTLVTVSS | |
| | ASTKGPSVFPLAPCSRSTSE | |
| | STAALGCLVKDYFPEPVTVS | |
| | WNSGALTSGVHTFPAVLQSS | |
| | GLYSLSSVVTVPSSNFGTQT | |
| | YTCNVDHKPSNTKVDKTVER | |
| | KCCVECPPCPAPPVAGPSVF | |
| | LFPPKPKDTLMISRTPEVTC | |
| | VVVDVSHEDPEVQFNWYVDG | |
| | VEVHNAKTKPREEQFNSTFR | |
| | VVSVLTVVHQDWLNGKEYKC | |
| | KVSNKGLPAPIEKTISKTKG | |
| | QPREPQVYTLPPSREEMTKN | |
| | QVSLTCLVKGFYPSDIAVEW | |
| | ESNGQPENNYKTTPPMLDSD | |
| | GSFFLYSKLTVDKSRWQQGN | |
| | VFSCSVMHEALHNHYTQKSL | |
| | SLSPGK | |
| Light | EIVMTQSPATLSVSPGERAT | 10 |
| Chain | LSCRASESVDNDGIRFLHWY | |
| | QQKPGQAPRLLIYRASTRAT | |
| | GIPARFSGSGSRTEFTLTIS | |
| | SLQSEDFAVYYCQQSNKDPY | |
| | TFGGGTKVEIKRTVAAPSVF | |
| | IFPPSDEQLKSGTASVVCLL | |
| | NNFYPREAKVQWKVDNALQS | |
| | GNSQESVTEQDSKDSTYSLS | |
| | STLTLSKADYEKHKVYACEV | |
| | THQGLSSPVTKSFNRGEC | |

In some aspects, the antibody molecule useful for the present methods comprises one, two, or three CDRs of the VH region and/or the VL region of an antibody molecule described herein, e.g., in Table 1 (e.g. Sibeprenlimab). In some aspects, the antibody molecule useful for the present methods comprises the VH region and/or the VL region of an antibody molecule described herein, e.g., in Table 1 (e.g. Sibeprenlimab). In some aspects, the antibody molecule useful for the present methods comprises the HC and/or the LC of an antibody molecule described herein, e.g., in Table 2 (e.g. Sibeprenlimab). In some aspects, the antibody molecule comprises a VH region having the amino acid sequence as set forth in SEQ ID NO: 1 and/or a VL region having the amino acid sequence as set forth in SEQ ID NO: 2. In some aspects, the antibody molecule comprises a HC having the amino acid sequence as set forth in SEQ ID NO: 9 and/or a LC having the amino acid sequence as set forth in SEQ ID NO: 10.

In some aspects, the antibody molecule further comprises a heavy chain constant region. In some aspects, the heavy chain constant region is an IgG1 constant region, or a functional portion thereof. In another aspect, the heavy chain constant region is an IgG2 constant region, or a functional portion thereof. In some aspects, the antibody molecule further comprises a light chain constant region. In some aspects, the antibody molecule further comprises a heavy chain constant region and a light chain constant region.

In some aspects, the antibody molecule useful for the present methods comprises: (i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising an amino acid sequence that differs by no more than 1, 2, or 3 amino acid residues from, or has at least 85, 90, 95, 99 or 100% homology with, the amino acid sequence of SEQ ID NO: 3; an HCDR2 comprising an amino acid sequence that differs by no more than 1, 2, or 3 amino acid residues from, or has at least 85, 90, 95, 99 or 100% homology with, the amino acid sequence of SEQ ID NO: 4; or an HCDR3 comprising an amino acid sequence that differs by no more than 1, 2, or 3 amino acid residues from, or has at least 85, 90, 95, 99 or 100% homology with, the amino acid sequence of SEQ ID NO: 5, or (ii) a light chain variable region (VL), wherein the light chain variable region comprises three light chain complementarity determining regions (LCDR1, LCDR2, and LCDR3), wherein the light chain variable region comprises: an LCDR1 comprising an amino acid sequence that differs by no more than 1, 2, or 3 amino acid residues from, or has at least 85, 90, 95, 99 or 100% homology with, the amino acid sequence of the LCDR1 of SEQ ID NO: 6; an LCDR2 comprising an amino acid sequence that differs by no more than 1, 2, or 3 amino acid residues from, or has at least 85, 90, 95, 99 or 100% homology with, the amino acid sequence of SEQ ID NO: 7; or an LCDR3 comprising an amino acid sequence that differs by no more than 1, 2, or 3 amino acid residues from, or has at least 85, 90, 95, 99 or 100% homology with, the amino acid sequence of SEQ ID NO: 8.

In some aspects, the antibody molecule useful for the present methods comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 3; an HCDR2 comprising the amino acid sequence of SEQ ID NO: 4; and an HCDR3 comprising the amino acid sequence of SEQ ID NO: 5, and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 6; an LCDR2 comprising the amino acid sequence of SEQ ID NO: 7; or an LCDR3 comprising the amino acid sequence of SEQ ID NO: 8.

In some aspects, the antibody molecule comprises a VH comprising an amino acid sequence having at least 85, 90, 95, 99 or 100% sequence identity to SEQ ID NO: 1. In some aspects, the antibody molecule comprises a VL comprising an amino acid sequence having at least 85, 90, 95, 99 or 100% sequence identity to SEQ ID NO: 2. In some aspects, the antibody molecule comprises a VH comprising the amino acid sequence as set forth in SEQ ID NO: 1. In some aspects, the antibody molecule comprises a VL comprising the amino acid sequence as set forth in SEQ ID NO: 2.

In some aspects, the antibody molecule useful for the present methods comprises:
(i) a heavy chain variable region (VH), wherein the heavy chain variable region comprises three heavy chain complementarity determining regions (HCDR1, HCDR2, and HCDR3), wherein the heavy chain variable region comprises: an HCDR1 comprising the amino acid sequence of SEQ ID NO: 3; an HCDR2 comprising the amino acid sequence of SEQ ID NO: 4; and an HCDR3 comprising the amino acid sequence of SEQ ID NO: 5, and wherein the VH comprises an amino acid sequence having at least 85, 90, 95, 99 or 100% sequence identity to SEQ ID NO: 1; and
(ii) a light chain variable region (VL), wherein the light chain variable region comprises three light chain complementarity determining regions (LCDR1, LCDR2, and LCDR3), wherein the light chain variable region comprises: an LCDR1 comprising the amino acid sequence of the LCDR1 of SEQ ID NO: 6; an LCDR2 comprising the amino acid sequence of SEQ ID NO: 7; or an LCDR3 comprising the amino acid sequence of SEQ ID NO: 8 and wherein the VL comprises an amino acid sequence having at least 85, 90, 95, 99 or 100% sequence identity to SEQ ID NO: 2.

In some aspects, the antibody molecule comprises a heavy chain (HC) comprising an amino acid sequence having at least 85, 90, 95, 99 or 100% sequence identity to SEQ ID NO: 9. In some aspects, the antibody molecule comprises a light chain (LC) comprising an amino acid sequence having at least 85, 90, 95, 99 or 100% sequence identity to SEQ ID NO: 10. In some aspects, the antibody molecule comprises a HC comprising the amino acid sequence as set forth in SEQ ID NO: 9 and a LC comprising the amino acid sequence as set forth in SEQ ID NO: 10.

In some aspects, the antibody molecule useful for the present methods has one or more (e.g., 2, 3, 4, 5, or all) of the following properties: (a) is a humanized antibody molecule; (b) binds to human APRIL at an EC₅₀ of 60 pM or less, as determined by ELISA; (c) inhibits binding of human APRIL to TACI, e.g., in vitro, at an IC₅₀ of 0.5 nM or less; (d) inhibits binding of human APRIL to BCMI, e.g., in vitro, at an IC₅₀ of 0.6 nM or less; (e) is an IgG2κ; or (f) has an Fc region engineered to reduce complement activation. In some aspects, the antibody molecule comprises one or more (e.g., 2, 3, 4, 5, or all) CDRs, one or both of heavy chain variable region or light chain variable regions, or one or both of heavy chain or light chain, of sibeprenlimab. In some aspects, the antibody molecule is suitable for use in treating Sjögren's disease.

In some aspects, the antibody molecule binds, or substantially binds, to an epitope that comprises one or more APRIL residues from the C-D loop (e.g., the loop connecting β-sheets C and D), the G-H loop (e.g., the loop connecting β-sheets G and H), or both. In some aspects, the antibody molecule binds, or substantially binds, to one or more (e.g., 2, 3, 4, 5, 6, 7, 8, 9, or all) residues of human APRIL from positions 105-114 and/or one or more (e.g., 2, 3, 4, 5, 6, 7, 8, 9, or all) residues of mouse APRIL from positions 96-105. In some aspects, the epitope is a conformational epitope.

In some aspects, the antibody molecule does not bind, or does not substantially bind, to one, two or all of Asp129, Arg233, or His203 of human APRIL.

In some aspects, binding of the antibody molecule to APRIL (e.g., human APRIL) inhibits, or substantially inhibits, the binding of the CRD2 domain of TACI (e.g., human TACI) to APRIL (e.g., human APRIL). In another aspect, binding of the antibody molecule to human APRIL, inhibits, or substantially inhibits, the binding of human TACI, to one or more, e.g., 2, 3, 4, 5, 6, 7, 8, 9, 10, 11, 12, 13, 14, 15, 16, 17, 18, 19, 20, 21, 22, 23, 24, 25, or all of the APRIL residues.

In some aspects, the antibody molecule useful for the present disclosure comprises Zigakibart/BION-1301. See WO 2010/100056, published Sep. 10, 2010; WO2015/034364, published Mar. 12, 2015; WO2016/110587, published Jul. 14, 2016; WO2021/243298, published Dec. 2, 2021; and WO2024/105549, published May 23, 2024, incorporated herein by reference in their entireties. In some aspects, an APRIL inhibitor useful for the present disclosure comprises Atacicept. See WO2002/094852, published Nov. 28, 2002. In some aspects, an APRIL inhibitor useful for the present disclosure comprises Povetacicept. See WO2021/226551, published Nov. 11, 2021; WO2022/236335, published Nov. 10, 2022; and WO2024/077018, published Apr. 11, 2024. In some aspects, an APRIL inhibitor useful for the present disclosure comprises a protein disclosed in WO2024/123675.

The present disclosure also provides a method of treating Sjögren's syndrome in a subject in need thereof or a population of subjects in need thereof comprising administering Zigakibart/BION-1301 in the subject or the population of subjects. In some aspects, Zigakibart/BION-1301 is administered at about 450 mg once every two weeks intravenously. In some aspects, Zigakibart/BION-1301 is administered at 600 mg Q2W once every two weeks subcutaneously. In some aspects, Zigakibart/BION-1301 is administered for two times, three times, four times, five times, six times, seven times, or eight times. In some aspects, the subject or each of the subjects after the administration of Zigakibart/BION-1301 (e.g., about 600 mg every two weeks subcutaneously) exhibits one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

The present disclosure also provides a method of treating Sjögren's syndrome in a subject in need thereof or a population of subjects in need thereof comprising administering Zigakibart/BION-1301 to the subject or the population of subjects. In some aspects, Zigakibart/BION-1301 is administered at about 450 mg once every two weeks intravenously. In some aspects, Zigakibart/BION-1301 is administered at 600 mg Q2W once every two weeks subcutaneously. In some aspects, Zigakibart/BION-1301 is administered for two times, three times, four times, five times, six times, seven times, eight times, ten times, 11 times, 12 times, 13 times, 14 times, 15 times, or 16 times. In some aspects, the subject or each of the subjects after the administration of Zigakibart/BION-1301 (e.g., about 600 mg every two weeks subcutaneously) exhibits one or more of the following:
a. a decrease in individual ESSDAI domains from the baseline at 52 weeks after the initial administration;
b. an increase in the salivary flow rate and/or tear flow rate, e.g., 0.1 mL/min or higher from the baseline at 52 weeks after the initial administration;
c. a decrease in Physician's global assessment (PhGA) score of disease activity from the baseline at 52 weeks after the initial administration;
d. a decrease in Patient's global assessment (PaGA) score of participant outcomes from the baseline at 52 weeks after the initial administration;
e. a decrease in Functional Assessment of Chronic Illness Therapy-Fatigue (FACIT-F) score from the baseline at 52 weeks after the initial administration;
f. an improvement in 36-Item Short Form Health Survey questionnaire version 2 (SF-36v2) physical and mental composite score from the baseline at 52 weeks after the initial administration; and
g. an improvement in Patient-reported Sjögren's disease diary from the baseline at 52 weeks after the initial administration.

The present disclosure also provides a method of treating Sjögren's syndrome in a subject in need thereof or a population of subjects in need thereof comprising administering Atacicept to the subject or a population of subjects. In some aspects, Atacicept is administered at about 75 mg or about 150 mg subcutaneously once weekly. In some aspects, Atacicept is administered for two times, three times, four times, five times, six times, seven times, or eight times. In some aspects, the subject or each of the subjects after the administration of Atacicept exhibits one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

The present disclosure also provides a method of treating Sjögren's syndrome in a subject in need thereof or a population of subjects in need thereof comprising administering Atacicept to the subject or the population of subjects. In some aspects, Atacicept is administered at about 75 mg or about 150 mg subcutaneously once weekly. In some aspects, Atacicept is administered for two times, three times, four times, five times, six times, seven times, eight times, ten times, 11 times, 12 times, 13 times, 14 times, 15 times, or 16 times. In some aspects, the subject or each of the subjects after the administration of Atacicept exhibits one or more of the following:
a. a decrease in individual ESSDAI domains from the baseline at 52 weeks after the initial administration;
b. an increase in the salivary flow rate and/or tear flow rate, e.g., 0.1 mL/min or higher from the baseline at 52 weeks after the initial administration;
c. a decrease in Physician's global assessment (PhGA) score of disease activity from the baseline at 52 weeks after the initial administration;
d. a decrease in Patient's global assessment (PaGA) score of participant outcomes from the baseline at 52 weeks after the initial administration;
e. a decrease in Functional Assessment of Chronic Illness Therapy-Fatigue (FACIT-F) score from the baseline at 52 weeks after the initial administration;
f. an improvement in 36-Item Short Form Health Survey questionnaire version 2 (SF-36v2) physical and mental composite score from the baseline at 52 weeks after the initial administration; and
g. an improvement in Patient-reported Sjögren's disease diary from the baseline at 52 weeks after the initial administration.

The present disclosure also provides a method of treating Sjögren's syndrome in a subject in need thereof or a population of subjects in need thereof comprising administering Povetacicept to the subject or the population of subjects. In some aspects, Povetacicept is administered for two times, three times, four times, five times, six times, seven times, or eight times. In some aspects, the subject or each of the subjects after the administration of Povetacicept exhibits one or more of the following:
a. a decrease in individual ESSDAI domains from the baseline at 28 weeks after the initial administration;
b. an increase in the salivary flow rate and/or tear flow rate, e.g., 0.1 mL/min or higher from the baseline at 28 weeks after the initial administration;
c. a decrease in Physician's global assessment (PhGA) score of disease activity from the baseline at 28 weeks after the initial administration;
d. a decrease in Patient's global assessment (PaGA) score of participant outcomes from the baseline at 28 weeks after the initial administration;
e. a decrease in Functional Assessment of Chronic Illness Therapy-Fatigue (FACIT-F) score from the baseline at 28 weeks after the initial administration;
f. an improvement in 36-Item Short Form Health Survey questionnaire version 2 (SF-36v2) physical and mental composite score from the baseline at 28 weeks after the initial administration; and
g. an improvement in Patient-reported Sjögren's disease diary from the baseline at 28 weeks after the initial administration.

The present disclosure also provides a method of treating Sjögren's syndrome in a subject in need thereof or a population of subjects in need thereof comprising administering Povetacicept to the subject or the population of subjects. In some aspects, Povetacicept is administered for two times, three times, four times, five times, six times, seven times, eight times, ten times, 11 times, 12 times, 13 times, 14 times, 15 times, or 16 times. In some aspects, the subject or each of the subjects after the administration of Povetacicept exhibits one or more of the following:
a. a decrease in individual ESSDAI domains from the baseline at 52 weeks after the initial administration;
b. an increase in the salivary flow rate and/or tear flow rate, e.g., 0.1 mL/min or higher from the baseline at 52 weeks after the initial administration;
c. a decrease in Physician's global assessment (PhGA) score of disease activity from the baseline at 52 weeks after the initial administration;
d. a decrease in Patient's global assessment (PaGA) score of participant outcomes from the baseline at 52 weeks after the initial administration;
e. a decrease in Functional Assessment of Chronic Illness Therapy-Fatigue (FACIT-F) score from the baseline at 52 weeks after the initial administration;
f. an improvement in 36-Item Short Form Health Survey questionnaire version 2 (SF-36v2) physical and mental composite score from the baseline at 52 weeks after the initial administration; and
g. an improvement in Patient-reported Sjögren's disease diary from the baseline at 52 weeks after the initial administration.

### Pharmaceutical Compositions and Kits

In some aspects, this disclosure provides compositions, e.g., pharmaceutically acceptable compositions, which include an antibody molecule described herein (e.g., a humanized antibody molecule described herein), formulated together with a pharmaceutically acceptable carrier that can be used to treat Sjögren's disease.

As used herein, “pharmaceutically acceptable carrier” or “pharmaceutically acceptable excipient” includes any and all solvents, dispersion media, isotonic and absorption delaying agents, and the like that are physiologically compatible. The carrier or excipient can be suitable for intravenous, intramuscular, subcutaneous, parenteral, rectal, spinal or epidermal administration (e.g., by injection or infusion). In some aspects, less than about 5%, e.g., less than about 4%, 3%, 2%, or 1% of the antibody molecules in the pharmaceutical composition are present as aggregates. In other aspects, at least about 95%, e.g., at least about 96%, 97%, 98%, 98.5%, 99%, 99.5%, 99.8%, or more of the antibody molecules in the pharmaceutical composition are present as monomers. In some aspects, the level of aggregates or monomers is determined by chromatography, e.g., high performance size exclusion chromatography (HP-SEC).

The compositions useful for the present methods may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some aspects, the antibody molecule is administered by intravenous infusion or injection. In some aspects, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m2, preferably about 5 to 50 mg/m², about 7 to 25 mg/m² and more preferably, about 10 mg/m². As will be appreciated by the skilled artisan, the route and/or mode of administration will vary depending upon the desired results. In certain aspects, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing Sjögren's disease. Alternatively, this property of a composition can be evaluated by examining the ability of the antibody molecule to inhibit APRIL, e.g., by an in vitro assay.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., Sjögren's disease, can be diagnosed in vitro, ex vivo, or in vivo.

In some aspects, the present disclosure provides a pharmaceutical composition comprising: (a) an anti-APRIL antibody and (b) one or more ingredients comprising L-arginine, L-glutamic acid, L-histidine, L-histidine monohydrochloride, polysorbate 80, and/or sorbitol. In some aspects the present disclosure provides a pharmaceutical composition comprising: (a) an anti-APRIL antibody and (b) one or more ingredients comprising L-arginine, L-glutamic acid, L-histidine, polysorbate 80, and/or sorbitol.

In some aspects, the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein: (a) the VH CDR1 comprises the amino acid sequence set forth in SEQ ID NO: 3; (b) the VH CDR2 comprises the amino acid sequence set forth in SEQ ID NO: 4; (c) the VH CDR3 comprises the amino acid sequence set forth in SEQ ID NO: 5; (d) the VL CDR1 comprises the amino acid sequence set forth in SEQ ID NO: 6; (e) the VL CDR2 comprises the amino acid sequence set forth in SEQ ID NO: 7; and (f) the VL CDR3 comprises the amino acid sequence set forth in SEQ ID NO: 8. In some aspects, the VH comprises the amino acid sequence as set forth in SEQ ID NO: 1. In some aspects, the VL comprises the amino acid sequence as set forth in SEQ ID NO: 2. In some aspects, the anti-APRIL antibody further comprises a constant region linked to the VH and or VL. In some aspects, the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody comprises zigakibart/BION-1301.

In some aspects, the pharmaceutical composition is a liquid formulation. In some aspects, the pharmaceutical composition is a liquid formulation for subcutaneous injection. In some aspects, the pharmaceutical composition is contained within a syringe. In some aspects, the pharmaceutical composition is contained within an autoinjector. In some aspects, the pH of the pharmaceutical composition is between about 5 and about 7, e.g., about 5.0, about 5.25, about 5.5, about 5.75, about 6.0, about 6.25, about 6.5, about 6.75, or about 7.0. In some aspects, the pH is about 6.2. In some aspects, the total volume of the composition is between about 1 and about 3 mL, e.g., about 1 mL, about 1.25 mL, about 1.5 mL, about 1.75 mL, about 2.0 mL, about 2.25 mL, about 2.5 mL, about 2.75 mL, or about 3.0 mL. In some aspects, the total volume of the composition is about 2 mL.

TABLE 3
The ingredients of an exemplary pharmaceutical
composition are provided below.

| Ingredients | Mass (mg) | Concentration | |
|---|---|---|---|
| L-arginine | 17.6 | 51 | mM |
| L-glutamic acid | 14.8 | 50 | mM |
| L-histidine | 4.3 | 14 | mM |
| L-histidine | 4.6 | 11 | mM |
| monohydrochloride | | | |
| Polysorbate 80 | 0.4 | 200 | mg/mL |
| Sorbitol | 36.4 | 100 | mM |

In some aspects, the pharmaceutical composition comprises the ingredients described herein, e.g., the ingredients described in Table 3. In some aspects, the pharmaceutical composition comprises about 400 mg of the anti-APRIL antibody, about 17.6 mg of L-arginine, about 14.8 mg of L-glutamic acid, about 4.3 mg of L-histidine, about 4.6 mg of L-histidine monohydrochloride, about 0.4 mg of polysorbate 80, and about 36.4 mg of sorbitol. In some aspects, the pharmaceutical composition further comprises water for injection. In some aspects, the pH of the composition is about 6.2. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol. In some aspects, the composition comprises about 200 mg/mL of the anti-APRIL antibody, about 51 mM L-arginine, about 50 mM L-glutamic acid, about 14 mM L-histidine, about 11 mM L-histidine monohydrochloride, about 200 mg/mL polysorbate 80, and about 100 mM sorbitol, and wherein the pH of the composition is about 6.2. In some aspects, the composition comprises about 200 mg/mL of the anti-APRIL antibody, about 51 mM L-arginine, about 50 mM L-glutamic acid, about 25 mM L-histidine, about 200 mg/mL polysorbate 80, and about 100 mM sorbitol. In some aspects, the pharmaceutical composition further comprises a trace amount of a chloride ion. In some aspects, the composition comprises between about 10 mg and about 1000 mg of the anti-APRIL antibody, e.g., about 10 mg, about 50 mg, about 100 mg, about 150 mg, about 200 mg, about 250 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 800 mg, about 900 mg, or about 1000 mg. In some aspects, the composition comprises about 400 mg of the anti-APRIL antibody. In some aspects, the pharmaceutical composition further comprises an overfill. In some aspects, the overfill comprises about 0.0125 mL to about 0.5 mL. In some aspects, the overfill comprises about 0.025 mL to about 0.5 mL, about 0.025 to about 0.05 mL, about 0.05 mL to about 0.075 mL, about 0.075 mL to about 0.1 mL, about 0.1 mL to about 0.125 mL, about 0.125 mL to about 0.15 mL, about 0.15 mL to about 0.175 mL, about 0.175 mL to about 0.2 mL, about 0.2 mL to about 0.225 mL, about 0.225 mL to about 0.25 mL, about 0.25 mL to about 0.275 mL, about 0.275 mL to about 0.3 mL, about 0.3 mL to about 0.325 mL, about 0.325 mL to about 0.35 mL, about 0.35 mL to about 0.375 mL, about 0.375 mL to about 0.4 mL, about 0.4 mL to about 0.425 mL, about 0.425 mL to about 0.45 mL, about 0.45 mL to about 0.475 mL, or about 0.475 mL to about 0.5 mL. In some aspects, the overfill comprises about 0.0125 mL, about, 0.025 mL, about 0.05 mL, about 0.075 mL, about 0.1 mL, about 0.125 mL, about 0.15 mL, about 0.175 mL, about 0.2 mL, about 0.225 mL, about 0.25 mL, about 0.275 mL, about 0.3 mL, about 0.325 mL, about 0.35 mL, about 0.375 mL, about 0.4 mL, about 0.425 mL, about 0.45 mL, about 0.475 mL, or about 0.5 mL.

In some aspects, the present disclosure provides a pharmaceutical composition comprising about 51 mM L-arginine, about 50 mM L-glutamic acid, about 14 mM L-histidine, about 11 mM L-histidine monohydrochloride, about 200 mg/mL polysorbate 80, about 100 mM sorbitol, and about 200 mg/mL of the anti-APRIL antibody, wherein the pH of the composition is about 6.2, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10.

In some aspects, the present disclosure provides a pharmaceutical composition comprising about 51 mM L-arginine, about 50 mM L-glutamic acid, about 25 mM L-histidine, about 200 mg/mL polysorbate 80, about 100 mM sorbitol, and about 200 mg/mL of the anti-APRIL antibody, wherein the pH of the composition is about 6.2, wherein the total volume of the composition is about 2 mL, and wherein the anti-APRIL antibody comprises a heavy chain (HC) comprising the amino acid sequence as set forth in SEQ ID NO: 9 and a light chain (LC) comprising the amino acid sequence as set forth in SEQ ID NO: 10. In some aspects, the anti-APRIL antibody in the composition is about 400 mg. In some aspects, the anti-APRIL antibody comprises Sibeprenlimab or an antigen binding portion thereof.

In some aspects, the present disclosure provides a method of treating a disease or disorder associated with APRIL, e.g., Sjögren's disease, in a subject in need thereof comprising administering to the subject the pharmaceutical composition.

In some aspects, the present disclosure provides a method of producing a pharmaceutical composition comprising admixing one or more ingredients in the pharmaceutical composition.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

### EXAMPLES

### Example 1: Anti-APRIL Antibody for the Treatment of Sjögren's Disease

### Trial Rationale:

An anti-APRIL antibody, 2419-1406, is used for a phase 2, multicenter, randomized, double-blind, placebo-controlled, proof-of concept trial followed by an optional open-label extension to evaluate the efficacy and safety of the antibody administered subcutaneously (SC) as an add-on to background treatment in participants with Sjögren's disease.

Screening evaluations will take place up to 30 days prior to the first dose of investigational medicinal product (IMP).

Up to 80 participants will be randomized in a 1:1 ratio to antibody or placebo (approximately 40 participants per treatment arm). Randomization will be stratified by screening disease activity score (ESSDAI <10 versus=10) and by type of background treatment at the time of randomization (no systemic therapy, any systemic therapy without steroids, or systemic therapy with steroids). The screening disease activity score will be assessed according to the ESSDAI.

The antibody or placebo will be administered as an SC injection once every 4 weeks. In the double-blind treatment period, 7 blinded doses will be administered, with Dose 1 administered on Day 1 and Dose 7 administered in Week 24. The primary assessment time point is Week 28. All participants will then be offered up to 7 additional doses of open-label antibody SC once every 4 weeks from Weeks 28 to 52 in the open-label extension period.

An interim efficacy analysis will be performed when approximately 50% of the participants either complete the Week 28 visit or prematurely discontinue from the trial.

The purpose of the interim analysis is to inform the assumptions of the phase 3, confirmatory trial(s). The interim analysis will not include formal hypothesis testing and is not intended to impact the trial conduct. While the interim analysis is conducted, participants will continue to enroll in the trial until the planned sample size of 80 participants are randomized.

Participants who discontinue treatment prior to Dose 7 will complete the Week 28 visit at the time of discontinuation and should complete the EOT visit within 8 weeks thereafter. For participants who complete the open-label extension, a follow-up visit will be conducted at Week 56 and the EOT visit will be conducted at Week 64, at which time final assessments will be performed.

### Trial Population:

Up to 80 participants (with the expectation of approximately 74 participants completing primary efficacy assessment) with Sjögren's disease who are between 18 and 75 years of age (inclusive) and of local legal consenting age at the time of signing the informed consent will be randomized in a 1:1 ratio to the antibody or placebo (approximately 40 participants in the antibody group and 40 in the placebo group).

### Key Inclusion/Exclusion Criteria:

Participants with Sjögren's disease according to the 2016 American College of Rheumatology (ACR)/European League Against Rheumatism (EULAR) criteria who have at least moderate disease activity (ESSDAI score=5), a stimulated salivary flow rate of =0.05 mL/min, and serum IgG >900 mg/dL. Participants are allowed to continue background treatments (as per local standard of care and applicable guidelines) that have been stable for at least 30 days at the time of randomization (hydroxychloroquine=400 mg/day, prednisone/prednisolone=10 mg/day, and/or one of the following: methotrexate=25 mg/week, leflunomide=20 mg daily, azathioprine=150 mg/day). Additionally, participants must be positive for anti-SSA (also referred to as anti-Ro) antibodies. Screening will include assessment for anti-Ro52 and anti-Ro60 and total IgG. Participants with another active autoimmune rheumatic disease, prior use of B-cell depleting therapy, or prohibited immunosuppressants will be excluded from participation in the trial. Participants with significant comorbidities including uncontrolled type 2 diabetes, malignancy, and chronic and/or acute infections will also be excluded.

### Trial Site(s):

Participants will be enrolled from approximately 50 global sites in 15 countries.

### Trial Intervention(s), Dose, Dosage Regimen, Treatment Duration, Formulation, Mode of Administration:

Investigational medicinal product will include antibody and placebo. The trial will evaluate a dose of 400 mg antibody compared with placebo. The IMP will be supplied as prefilled syringes (PFS) and will be administered SC once every 4 weeks as 7 doses of double-blind IMP from Day 1 to Week 24. All participants will then be offered up to 7 doses of open-label antibody SC from Weeks 28 to 52.

Healthcare professionals with training and experience with SC drug delivery can be designated by the investigator to administer the IMP. The IMP administration will be administered in the abdomen, thigh, or upper arm. The date and time of IMP administration, the volume of IMP administered, and the injection site location will be recorded for each IMP administration.

Participants are allowed to continue background treatment alone or in combination, though the dose must be stable for at least 30 days prior to randomization. During the trial, the dose of the background therapies must remain stable; reductions in dosing are permitted.

### Trial Assessments:

Assessments for Efficacy: The ESSDAI, ClinESSDAI, EULAR Sjögren's Syndrome Patient-Reported Index (ESSPRI), Sjögren Tool for Assessing Response (STAR), 36-Item Short Form Survey Version 2 (SF-36v2), Functional Assessment of Chronic Illness Therapy-Fatigue (FACIT-Fatigue), Physician Global Assessment, and Patient Global Assessment. Laboratory assessments required to derive hematological and biological domains of the ESSDAI and STAR will be performed on blood specimens at a central laboratory. Additional assessments for scoring the ESSDAI and STAR include neurologic examination, muscular examination, nerve conduction (only for participants with activity in the muscular or PNS domains of the ESSDAI/ClinESSDAI), electromyography (EMG; only for participants with activity in the muscular domain of the ESSDAI/ClinESSDAI), pulmonary function testing, and chest imaging.

Assessments for Pharmacokinetics (PK): Blood sampling for sibeprenlimab serum concentrations.

Assessments for Pharmacodynamics (PD): Blood sampling for APRIL, IgG, IgA, and IgM concentrations.

Assessments for Antibodies: Blood sampling for antidrug antibodies (ADA), including neutralizing antibodies, and autoantibodies characteristic of Sjögren's disease (including anti-SSA, anti-Sjögren's syndrome-related antigen B [anti-SSB], and rheumatoid factor [RF]).

Assessments for Safety: Adverse events (AEs), clinical laboratory assessments (serum chemistry, hematology, urinalysis), physical examination, vital signs, 12-lead electrocardiogram (ECG), assessment of injection site. Assessments for Exploratory Biomarkers: Exploratory biomarkers may include immunoglobulins, immune complexes, immune repertoire, inflammatory factors, and gene expression signatures over time. Additionally, stimulated and unstimulated salivary flow and tear flow rate will be collected.

Screening/Other: Medical history, pregnancy testing (participants of childbearing potential only), follicle stimulating hormone (FSH; perimenopausal and postmenopausal participants only), lymphocyte subsets, and samples for future biomarker research (FBR; optional). An eDiary will be employed to collect daily Sjögren's disease symptoms, a weekly impact questionnaire, and use of eye drops, lotion, lozenges, and mouthwash. A subset of participants will be asked to wear a digital wearable device for approximately 23 hours per day for the first 6 months of the trial to passively collect activity data and to actively collect data on daily eye drop use. An optional, site-dependent biopsy of the labial salivary gland may be performed at a subset of clinical sites for participants who provide consent and will be analyzed by histopathology and gene expression analyses.

### Statistical Methods:

General Considerations: All data collected will be documented using summary tables, figures, and/or data listings presented by treatment group.

Sample Size: Up to 80 participants will be randomized in a 1:1 ratio to receive treatment with the antibody or placebo (40 participants in the antibody group and 40 participants in the placebo group). The sample size is based on a t-test with a 2-sided type I error rate of 0.2. With 80 participants, the power will range between 76% and 90% to detect a 3-point treatment difference in change from baseline in ESSDAI at Week 28 between the antibody and placebo groups, assuming a common standard deviation (SD) ranging between 6.5 and 5. The sample size accounts for a nonevaluability/dropout rate of approximately 7% (up to a maximum of 6 participants) prior to Week 28.

Randomization and Stratification: Participants will be randomly assigned in a 1:1 ratio to receive treatment with the antibody or placebo. Randomization will be stratified by baseline ESSDAI score (<10 versus=10) and by type of background treatment at the time of randomization (no systemic therapy, any systemic therapy without steroids, or any systemic therapy with steroids).

Planned Analyses: The primary question of interest is the treatment difference in change from baseline at Week 28 in ESSDAI total score, in participants with Sjögren's disease, between the antibody and placebo as add-on therapy to background treatment regardless of IMP discontinuation. The primary estimand is described by the following attributes:
Population: randomized participants with Sjögren's disease who have a baseline value for the ESSDAI total score.
Variable: Change from baseline in ESSDAI total score at Week 28.
Intercurrent event 1 (ICE1): IMP discontinuation prior to Week 28. A treatment policy strategy will be followed to handle this ICE. As such, the sponsor will make every effort to collect efficacy assessments as planned even after IMP discontinuation.
Intercurrent event 2 (ICE2): Use of rescue therapies prior to Week 28, including new or intensification of existing background treatment. A treatment policy strategy will be followed to handle this ICE. The sponsor will make every effort to collect efficacy assessments as planned even after the use of rescue therapies.
Population-level summary: Difference in mean change from baseline in ESSDAI total score at Week 28 visit between the antibody and placebo arms. Missing data due to an intercurrent event or other reasons will be considered missing at random (MAR) for the main estimator.

The main estimate for this estimand will be obtained using a mixed model for repeated measurements (MMRM). The model will include the fixed effects of treatment, visit, treatment-by-visit interaction, as well as the type of background treatment at baseline (no systemic therapy, systemic therapy without steroids or systemic therapy with steroids) and the continuous covariate of baseline ESSDAI score. The within-participant correlation will be modeled using the unstructured covariance matrix. The difference of adjusted means between the antibody and placebo arms will be reported along with its corresponding 80% CI and p-value.

A similar estimand is defined for ESSPRI and a similar MMRM model will be used to analyze change from baseline in ESSPRI. The difference of adjusted means between the antibody and placebo arms will be reported for ESSPRI along with its corresponding 95% CI.

Comparisons between the antibody and placebo arms will be performed using MMRM for the other longitudinal continuous secondary efficacy endpoints.

The binary secondary efficacy endpoints, for example, the proportion of participants achieving clinically meaningful improvement in ESSDAI from baseline to primary time point, will be analyzed using an exact Cochran-Mantel-Haenszel (CMH) test stratified by randomization strata. The difference in proportions between the antibody group and placebo arms will be reported with the associated 95% CI. A missing responder value at primary time point due to discontinuation or for other reasons will be imputed as a nonresponder.

Safety will be assessed by evaluations of AEs, clinical laboratory tests, vital sign measurements, 12-lead ECG, physical examinations, injection site reactions, neurologic examination, muscular examination, nerve conduction, EMG, pulmonary function testing, and chest imaging.

Exploratory pharmacodynamic analyses will be conducted; data will be summarized over time by treatment group using descriptive statistics as appropriate. The antibody serum concentration data will be summarized over time by treatment visit.

Other assessments supporting the exploratory and/or other objectives will be summarized.

Interim Analysis: The interim efficacy analysis will be performed when approximately 50% of the participants either complete the Week 28 visit or prematurely discontinue from the trial. The purpose of the interim analysis is to inform the assumptions for a fully powered, phase 3, confirmatory trial(s). The interim analysis will not include formal hypothesis testing and is not intended to impact the trial conduct. While the interim analysis is conducted, participants will continue to enroll in the trial until the planned sample size of 80 participants are randomized.

A limited number of the unblinded personnel, independent of the trial team, may prepare group unblinded efficacy and exploratory summaries as well as Ig-related analysis (with individual participant treatment blinded) for the sponsor review. A small number of sponsor members who are not involved in the day-to-day conduct of the trial will review the group unblinded data. As the interim analysis only includes the first 50% of the planned enrolled participants, the rest of the participants will remain blinded.

To account for multiple reviews of the primary efficacy endpoint, the type I error rate applied will be reduced by 0.001 for the interim efficacy review, resulting in a 2-sided final analysis type I error rate of 0.199.

### Trial Duration:

Trial duration for each participant is expected to be approximately 68 weeks (assuming up to 30 days for screening, 52 weeks of trial treatment administered once every 4 weeks from Day 1 to Week 24 (double-blind treatment) and from Week 28 to Week 52 (open-label extension), a follow-up visit in Week 56, and ending with an end-of-trial visit in Week 64). Participants who discontinue treatment early will be encouraged to remain in the trial for safety follow-up. The trial start is defined as the date the first participant signs their informed consent form. Overall, the trial duration, from signing of the first participant's informed consent form to the last participant's final assessment, is expected to be approximately 28 months.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific aspects of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating Sjögren's disease in a subject in need thereof comprising administering to the subject an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain complementarity determining region (VL CDR)1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

2. A method of treating Sjögren's disease in a population of subjects in need thereof comprising administering to the subjects an anti-APRIL antibody or an antigen-binding portion thereof, wherein the anti-APRIL antibody comprises a heavy chain variable domain comprising a heavy chain complementarity determining region (VH CDR) 1, a VH CDR2, and a VH CDR3 and a light chain variable domain comprising a light chain (VL) CDR1, a VL CDR2, and a VL CDR3, wherein:
a) the VH CDR1 comprises the amino acid sequence set forth in SEQ ID NO: 3;
b) the VH CDR2 comprises the amino acid sequence set forth in SEQ ID NO: 4;
c) the VH CDR3 comprises the amino acid sequence set forth in SEQ ID NO: 5;
d) the VL CDR1 comprises the amino acid sequence set forth in SEQ ID NO: 6;
e) the VL CDR2 comprises the amino acid sequence set forth in SEQ ID NO: 7; and
f) the VL CDR3 comprises the amino acid sequence set forth in SEQ ID NO: 8.

3. The method of claim 1, wherein
(i) the VH comprises the amino acid sequence as set forth in SEQ ID NO: 1,
(ii) the VL comprises the amino acid sequence as set forth in SEQ ID NO: 2; or
(iii) the VH comprises the amino acid sequence as set forth in SEQ ID NO: 1 and the VL comprises the amino acid sequence as set forth in SEQ ID NO: 2.

4-5. (canceled)

6. The method of claim 1, wherein the anti-APRIL antibody comprises
(i) a heavy chain (HC) comprising the amino acid sequence as set forth in SEQ ID NO: 9;
(ii) and a light chain (LC) comprising the amino acid sequence as set forth in SEQ ID NO: 10; or
(iii) any combination of (i) and (ii).

7-14. (canceled)

15. The method of claim 1, wherein, before the administration, the subject (i) has a European League against Rheumatism (EULAR) Sjögren's syndrome disease activity index (ESSDAI) score equal to or higher than 1, 2, 3, 4, 5, 6, 7, 8, 9, 10, 11, 12, 13, 14, 15, 16, 17, 18, 19, or 20; (ii) is seropositive for an anti-Sjögren's-syndrome-related antigen A (anti-SSA/Ro) antibody, an anti-Sjögren's-syndrome-related antigen B (anti-SSB) antibody, or rheumatoid factor; (iii) has a stimulated whole salivary flow rate of equal to or higher than 0.05 mL/min; (iv) has a serum IgG concentration greater than 900 mg/dL; or (v) any combination thereof.

16-21. (canceled)

22. The method of claim 15, wherein the anti-SSA/Ro antibody comprises an anti-Ro52 antibody or anti-Ro60 antibody.

23-24. (canceled)

25. The method of claim 1, wherein the anti-APRIL antibody or the antigen-binding portion thereof or the APRIL inhibitor is administered (i) subcutaneously; (ii) at a fixed dose between 10 mg and 1000 mg; or (iii) twice, three times, four times, five times, six times, seven times, eight times, nine times, 10 times, 11 times, 12 times, 13 times, 14 times, or 15 times; (iv) for at least four weeks, at least 8 weeks, at least 12 weeks, at least 16 weeks, at least 20 weeks, at least 24 weeks, at least about 28 weeks, at least 32 weeks, at least 36 weeks, at least 40 weeks, at least 44 weeks, at least 48 weeks, at least 52 weeks, at least 56 weeks, at least 60 weeks, at least 64 weeks, at least 68 weeks, or at least 72 weeks; or (v) any combination thereof.

26. (canceled)

27. The method of claim 1, wherein the anti-APRIL antibody or the antigen-binding portion thereof or the APRIL inhibitor is administered at a fixed dose of 400 mg once every 4 weeks.

28-29. (canceled)

30. The method of claim 1, further comprising administering to the subject or each of the subjects a standard of care (SOC) therapy.

31. The method of claim 1, wherein the subject or each of the subjects received a SOC therapy prior to the administration.

32. The method of claim 31, wherein the subject or each of the subjects received a SOC therapy for at least 30 days prior to the administration.

33. (canceled)

34. The method of claim 1, wherein the SOC therapy comprises hydroxychloroquine, a corticosteroid (e.g., prednisone or prednisolone), methotrexate, leflunomide, azathioprine, or any combination thereof.

35-50. (canceled)

51. The method of claim 1, wherein after the administration, the subject exhibits one or more of the following:
a) a decrease in individual EULAR Sjögren's syndrome disease activity index (ESSDAI) domains from the baseline at 28 and/or 52 weeks after the initial administration;
b) an increase in the salivary flow rate and/or tear flow rate, e.g., 0.1 mL/min or higher from the baseline at 28 and/or 52 weeks after the initial administration;
c) a decrease in Physician's global assessment (PhGA) score of disease activity from the baseline at 28 and/or 52 weeks after the initial administration;
d) a decrease in Patient's global assessment (PaGA) score of participant outcomes from the baseline at 28 and/or 52 weeks after the initial administration;
e) a decrease in Functional Assessment of Chronic Illness Therapy-Fatigue (FACIT-F) score from the baseline at 28 and/or 52 weeks after the initial administration;
f) an improvement in 36-Item Short Form Health Survey questionnaire version 2 (SF-36v2) physical and mental composite score from the baseline at 28 and/or 52 weeks after the initial administration;
g) an improvement in Patient-reported Sjögren's disease diary from the baseline at 28 and/or 52 weeks after the initial administration,
h) a EULAR Sjögren's Syndrome Patient Reported Index ESSPRI that is equal to or higher than 1 point from the baseline at 28 and/or 52 weeks after the initial administration;
i) a reduction in total serum IgG;
i) a reduction in circulating peripheral immune cells;
k) a reduction in total serum IgA;
1) a reduction in total serum IgM;
m) a reduction in mononuclear cells staining positive for IgA, IgG, and/or IgM in Gut-associated lymphoid tissue (GALT) and/or tonsils;
n) a change in free APRIL concentrations;
o) a change in immunoglobulins characteristic of Sjögren's B-cell receptor;
p) a change in B-cell receptor repertoire;
q) a change in inflammatory factors; and
r) a change in gene expression signatures.

52. The method of claim 1, wherein the subject exhibits a decrease in serum anti-drug antibody (ADA).

53. The method of claim 1, wherein the treatment is terminated if the subject exhibits an increase in serum ADA or no decrease in serum ADA.

54-67. (canceled)

68. The method of claim 1, wherein the subject:
a) does not have another active autoimmune rheumatic disease;
b) has not received a B-cell depleting therapy or if received, the B-cell count of the subject or each of the subjects is less than the lower limit of normal or baseline value prior to the receipt of previous B cell-depleting therapy (whichever is lower);
c) does not exhibit active viral, bacterial, or other infections requiring systemic treatment at the time of screening or randomization, or history of recurrent clinically significant infection;
d) does not have history of a previous hypersensitivity or severe allergic reaction with generalized urticaria, angioedema, or anaphylaxis to the anti-APRIL antibody, L-arginine, L-glutamic acid, L-histidine, L-histidine monohydrochloride, polysorbate 80, and/or sorbitol;
e) has not had a history of major organ, hematopoietic stem cell, or bone marrow transplant;
f) does not take a medication known to cause dry mouth/eyes as a regular and major side effect, and which have not been on a stable dose for at least 30 days prior to the administration, or any anticipated change in the treatment regimen during the course of the administration;
g) does not use a topical ocular prescription medication (excluding artificial tears, gels, lubricants) that have not been on a stable dose for at least 90 days prior to administration, or any anticipated change in the treatment regimen during the course of the administration;
h) does not have a history of primary or secondary immunodeficiency, including a positive human immunodeficiency virus (HIV) test result;
i) has not a history of malignancy of any organ system (other than localized basal cell carcinoma of the skin or in situ cervical cancer), treated or untreated, within the past 5 years, regardless of whether there is evidence of local recurrence or metastases;
j) does not have sarcoidosis;
k) does not have a history of fibromyalgia;
l) does not have uncontrolled type 2 diabetes, as evidenced by a screening hemoglobin A1c (HbA1c) value >8%;
m) does not have a surgical, medical (eg, uncontrolled hypertension, heart failure, cerebrovascular accident), psychiatric or additional physical condition that interferes with the administration;
n) does not have a positive serology for hepatitis B surface antigen (HBsAg);
o) does not have hepatitis C;
p) does not have positive hepatitis C antibody and hepatitis C virus (HCV)-ribonucleic acid (RNA) at the administration or prior to administration except if the subject or each of the subjects has chronic hepatitis C, but has completed HCV anti-viral treatment and is HCV-RNA negative at least 12 weeks after the anti-viral treatment before the administration;
q) does not have active tuberculosis infection;
r) is not pregnant or nursing (lactating); or
s) any combination of a) through r).

69-70. (canceled)

71. The method of claim 1, wherein the subject has not been previously treated with one or more of the following within at least 6 months prior to the administration:
a) belimumab, abatacept, anti-tumor necrosis factor alpha biologic agents,
b) immunoglobulins, plasmapheresis, intravenous (IV) or oral cyclophosphamide; and
c) mycophenolate mofetil, IV or oral cyclosporine A, or any other immunosuppressants.

72. The method of claim 1, wherein the subject, before the administration, does not exhibit the following laboratory values:
a) Hemoglobin levels <8.0 g/dL;
b) White blood cells (WBC) count <2.0×103/μL;
c) Platelet count <100×103/μL;
d) Absolute neutrophil count (ANC)<0.8×103/μL; and
e) Estimated glomerular filtration rate (eGFR) calculated using the 2021 Chronic Kidney Disease-Epidemiology Collaboration (CKD-EPI) serum creatinine eGFR formula <45 mL/min/1.73 m2.

73-88. (canceled)

89. The method of claim 1, wherein, after the administration, the subject exhibits:
a) mean AUCtau of about 4000 day*μg/ml to about 6000 day*μg/ml, e.g., 4000 day*μg/ml, 4100 day*μg/ml, 4200 day*μg/ml, 4300 day*μg/ml, 4400 day*μg/ml, 4500 day*μg/ml, 4600 day*μg/ml, 4700 day*μg/ml, 4800 day*μg/ml, 4900 day*μg/ml, 5000 day*μg/ml, 5100 day*μg/ml, 5200 day*μg/ml, 5300 day*μg/ml, 5400 day*μg/ml, 5500 day*μg/ml, 5600 day*μg/ml, 5700 day*μg/ml, 5800 day*μg/ml, 5900 day*μg/ml, or 6000 day*μg/ml;
b) mean (SD) Cmax of about 100 μg/ml to about 300 Ug/ml, e.g., 110 μg/ml, 120 μg/ml, 130 μg/ml, 140 μg/ml, 150 μg/ml, 160 μg/ml, 170 μg/ml, 180 μg/ml, 190 μg/ml, 200 μg/ml, 210 μg/ml, 220 μg/ml, 230 μg/ml, 240 μg/ml, 250 μg/ml, 260 μg/ml, 270 μg/ml, 280 μg/ml, 290 μg/ml, 300 μg/ml;
c) mean Ctrough of about 100 μg/ml to about 200 μg/ml, e.g., 110 μg/ml, 120 μg/ml, 130 μg/ml, 140 μg/ml, 150 μg/ml, 160 μg/ml, 170 μg/ml, 180 μg/ml, 190 μg/ml or 200 μg/ml;
d) mean (SD) Ctrough of about 141 (94) Ig/mL, mean (SD) Cmax of about 194(100) μg/ml, mean (SD) AUCtau of about 4830 (2780) day*μg/mL; or
e) any combination of a) through d).

90-95. (canceled)

96. A method of treating Sjögren's disease in a subject in need thereof comprising administering to the subject an anti-APRIL antibody, wherein the anti-APRIL antibody comprises Sibeprenlimab or an antigen binding portion thereof.
